(19) United States
(12) Patent Application Publication
(10) Pub. No.: US 2026/0201017 A1
(43) Pub. Date: Jul. 16, 2026

(54) RECOMBINANT EXPRESSION VECTOR FOR PRODUCING HIGHLY-EXPRESSED HUMAN SERUM ALBUMIN, HOST STRAIN, AND USE OF HOST STRAIN

(71) Applicant: HEBEI HUAKAI HUIHE BIOMEDICAL CO., LTD., Baoding (CN)

(72) Inventors: Zhimin LIU, Baoding (CN); Kanghua WU, Baoding (CN)

(73) Assignee: HEBEI HUAKAI HUIHE BIOMEDICAL CO., LTD., Baoding (CN)

(21) Appl. No.: 19/634,119

(22) Filed: Mar. 31, 2026

Related U.S. Application Data
(63) Continuation-in-part of application No. PCT/CN2025/114086, filed on Aug. 12, 2025.

(30) Foreign Application Priority Data
Aug. 13, 2024 (CN) 202411107941.5

(51) Int. Cl. C07K 14/765; C12N 1/165; C12N 9/02; C12N 9/04; C12N 9/10; C12N 15/81; C12R 1/84

(52) U.S. Cl. CPC C07K 14/765; C12N 1/165; C12N 9/0006; C12N 9/0057; C12N 9/1022; C12N 15/81; C12R 2001/84; C12Y 101/01023; C12Y 110/03011; C12Y 202/01003

(57) ABSTRACT

A recombinant expression vector for producing highly expressed human serum albumin (HSA), a host strain, and a use of the host strain are provided. Through the optimization design for the target gene, the construction of a Pichia pastoris engineered strain carrying the exogenous gene with a high copy number and expressing the exogenous gene at a high level, and the optimization of a pilot-scale fermentation process, an expression level of the recombinant HSA can be as high as 25.82 g/L. A purification process for the recombinant HSA involves only fermentation broth pretreatment and three-step column chromatography, and enables a purity of 99% or more for the target protein. A recovery rate of the entire purification process reaches 60% or more.

### CROSS REFERENCE TO THE RELATED APPLICATIONS

This application is a continuation-in-part application of International Application No. PCT/CN2025/114086, filed on Aug. 12, 2025, which is based upon and claims priority to Chinese Patent Application No. 202411107941.5, filed on Aug. 13, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named ZDSH0502P_SequenceListing.xml, created on Feb. 11, 2026, and is 24,421 bytes in size.

### TECHNICAL FIELD

The present patent belongs to the biotechnology field of preparing human serum albumin (HSA) based on genetic engineering, and specifically relates to a recombinant expression vector for producing highly-expressed HSA, a host strain, and a use of the host strain.

### BACKGROUND

The development of molecular biotechnology has provided numerous approaches and means for preparing exogenous proteins with bioreactors. So far, various expression systems for exogenous proteins have been developed, including Escherichia coli (E. coli), yeast, insect, and mammalian cell systems. The Pichia pastoris gene expression system, after nearly three decades of development, has become one of the most important hosts for expressing exogenous proteins, such as the host strain GS115 (Cregg, et al. (2009). Methods Enzymol. 463, 169-189; U.S. Pat. No. 4,879,231, Phillips Petroleum, 1989). The Pichia pastoris gene expression system offers advantages such as easy high-density fermentation, stable integration of target genes into the host genome, effective secretion and appropriate glycosylation of expressed products, and cost-effective media. Through the highly-efficient and regulatable AOX1 promoter, the Pichia pastoris gene expression system has achieved the high-level expression of thousands of exogenous proteins, including hepatitis B surface antigen (HBsAg), tumor necrosis factor (TNF), epidermal growth factor (EGF), tetanus toxin fragment C, and genetically engineered antibodies. It has been proved that this gene expression system is efficient, practical, and simple, is predominantly characterized by the enhancement of expression levels and the retention of biological activities of products, and is highly suitable for pilot-scale amplification and large-scale industrial production.

The expression of exogenous genes in Pichia pastoris typically includes the following steps: (1) An exogenous gene is inserted into a Pichia pastoris expression vector to construct a recombinant expression vector. (2) The recombinant expression vector is digested and treated with restriction endonucleases to obtain a linearized recombinant plasmid and then transformed into a Pichia pastoris strain. (3) A transformant solution is coated on MD plates for the first round of screening of positive recombinants. (4) The second round of screening of positive recombinants is conducted with YPD plates including geneticin G418 at different concentrations. (5) The integration of the exogenous gene into the yeast genome is further identified. (6) An expression level of the exogenous gene is determined through small-scale induced expression. (7) Large-scale fermentation production is conducted with a bioreactor, and a recombinant protein is extracted from a fermentation broth. To improve the expression level of an exogenous gene in Pichia pastoris, it is usually necessary to select Pichia pastoris transformants carrying the exogenous gene with a high copy number, that is, the gene dosage in the host strain increases. The selection of high-copy-number transformants is achieved by progressively elevating the resistance to G418, which is inherently highly tedious and random. The further investigation shows that the relationship between high-copy-number Pichia pastoris transformants and highly-expressed exogenous proteins remains variable. Because the optimal copy number varies depending on the target gene, there is no linear relationship between high-copy-number Pichia pastoris transformants and highly-expressed exogenous proteins. In the series of recombinant expression vectors such as pPIC9K and pPIC3.5K commonly used for Pichia pastoris, a G418 resistance gene has been incorporated. After these recombinant expression vectors are transformed into Pichia pastoris, both an exogenous gene and the resistance gene are integrated into a chromosome of Pichia pastoris through homologous recombination. By increasing the concentration of G418 for screening, transformants that carry multi-copy insertions of the target gene and represent a minority in a transformant population can be selected from the transformant population.

To select positive clones carrying the exogenous gene with a high copy number, the screening needs to be conducted with G418 gradient plates with different concentrations. In the preliminary experiment, G418 concentrations are set to 0 mg/mL, 0.25 mg/mL, 0.5 mg/mL, 0.75 mg/mL, 1.0 mg/mL, 1.5 mg/mL, 1.75 mg/mL, 2.0 mg/mL, 3.0 mg/mL, and 4.0 mg/mL. According to the protocol (Pichia Expression Kit: A Manual of Methods for Expression of Recombinant Proteins in Pichia pastoris, Catalog No. K1710-01) recommended by Invitrogen, the following screening methods are described: 1. G418 gradient plates with different concentrations are prepared, and positive clones are then transferred from an HIS4 auxotrophic plate one by one to these G418 gradient plates through replica plating to achieve the screening of high-copy-number clones. This method is characterized by immense workload (three consecutive subcultures are required before replica plating to ensure equivalent densities for individual transformants), high screening difficulty, prolonged cycle, and relatively-limited number of screened transformants, making it unsuitable for high-throughput screening. 2. G418 gradient plates with different concentrations are prepared, and all positive transformants growing on an HIS4 auxotrophic plate can be washed off with sterile water or a liquid medium, then diluted to an appropriate concentration, and then coated at a specified density on the G418 gradient plates with different concentrations. This method involves relatively simple operations and enables the screening of a larger number of transformants. However, for different target genes or different Pichia pastoris host strains, a preliminary experiment is required to determine the optimal dilution factor for a strain suspension. The above two screening methods are currently the universal screening methods employed by scientific researchers worldwide in the production of exogenous proteins with the Pichia pastoris expression system. However, both of the two screening methods demonstrate drawbacks including high G418 consumption, cumbersome procedures, heavy workload, high time and labor consumption, and poor universality across different Pichia pastoris host strains.

### SUMMARY

### 1. Technical Problem to be Solved by the Present Disclosure

An objective of the present patent is to address the following problems: The existing technique for preparing HSA through genetic recombination has drawbacks such as low gene expression levels and immature fermentation process control (due to the large scale of fermentation, 10-ton or dozens-of-ton large fermenters are often required, and there is a lack of precedents and tools for process control and monitoring means for large fermenters). The existing purification process for recombinant HSA is immature (since recombinant HSA is a special case, the purification scale and equipment for the target protein are exceptionally large, and the target protein needs to have an extremely-high purity of 99.99% or more). That is, there are problems such as tens of purification steps of the recombinant protein, low purification medium load, low purification efficiency, and low purification recovery rate.

### 2. Technical Solution

In order to achieve the above objective, the present disclosure provides the following technical solutions:

The present disclosure provides a recombinant expression vector for producing highly-expressed HSA, where a nucleotide sequence of the recombinant expression vector is shown in SEQ ID NO: 3.

Preferably, the recombinant expression vector includes:
(1) a 5′ regulatory region that is derived from Pichia pastoris and includes a promoter element, where the 5′ regulatory region is selected from a 5′ regulatory region of an alcohol oxidase 1 (AOX1) gene, a dihydroxyacetone synthase (DAS1) gene, or a histidinol dehydrogenase (HIS4) gene that is derived from the Pichia pastoris, and the 3′ terminus of the 5′ regulatory region is linked to a sequence described in (2) below;
(2) an optimized gene encoding the HSA, where a nucleotide sequence of the optimized gene is shown in SEQ ID NO: 1, and an amino acid sequence of the HSA is shown in SEQ ID NO: 2; and
(3) a 3′ termination sequence derived from the Pichia pastoris, where the 3′ termination sequence is selected from a 3′ termination sequence of the AOX1 gene, an alcohol oxidase 2 (AOX2) gene, or the HIS4 gene that is derived from the Pichia pastoris.

Preferably, the recombinant expression vector further includes:
at least one marker gene for screening in E. coli, such as the ampicillin resistance gene (Amp^{R});
a DNA fragment of a replication origin capable of replicating in an E. coli host strain, such as pBR322 Ori; and
at least two marker genes for screening in a yeast, such as Kan^{R} and HIS4.

Preferably, the recombinant expression vector is a linearized vector produced by cleaving any one of pPIC9, pPIC3, pPICZαA, B and C, pPIC3.5K, pHIL-S1, pHIL-D2, pA0804, pA0815, pGAPZαABC, pPIC6αABC, and pPIC9K with a restriction endonuclease SacI or BglII.

Preferably, the recombinant expression vector is a linearized vector produced by cleaving HSA-pPIC9K with the restriction endonuclease SacI or BglII, and the linearized vector includes at least one copy of the optimized gene encoding the HSA.

Preferably, in the optimized gene encoding the HSA, optimal codons preferred by the AOX1 gene derived from the Pichia pastoris are adopted, and a proportion of the optimal codons in total codons of the optimized gene is controlled at 90%.

Preferably, three restriction endonuclease sites SalI, HindIII, and XbaI are inserted sequentially in a 5′ to 3′ direction within the optimized gene encoding the HSA, such that the optimized gene is divided into four relatively-balanced large fragments.

Preferably, in the optimized gene encoding the HSA, consecutive G-C base pairs are reduced and A-T base pairs preferred by the Pichia pastoris are increased, achieving a balanced design of the nucleotide distribution within the optimized gene, such that a GC content in the optimized gene is adjusted to 45% to 50%.

Preferably, the optimized gene encoding the HSA further includes an expression reading cassette constructed as follows:
inserting a 5′ restriction endonuclease site BamHI into a 5′ regulatory region (promoter region) of the AOX1 gene, ligating a 10-deoxynucleotide oligonucleotide CCAAACGATG as shown in SEQ ID NO: 9 (including a Kozak sequence for eukaryotic genes, which is AXXATG), ligating a yeast α-mating factor leader peptide sequence (including 85 amino acids) derived from Saccharomyces cerevisiae, and inserting a mature HSA gene between multiple cloning sites EcoRI and NotI of a pPIC9K recombinant expression vector; and
inserting a coding sequence AAAAGA for a dibasic amino acid of lysine and arginine (-Lys-Arg-) after an enzyme cleavage site EcoRI at a 5′ terminus of the mature HSA gene, where the specific sequence is as shown in SEQ ID NO: 4, and inserting a double stop codon TAATAG before an enzyme cleavage site NotI at a 3′ terminus of the mature HSA gene, where the specific sequence is as shown in SEQ ID NO: 5.

A host strain for high-level expression of HSA is provided, where the host strain is Pichia pastoris HSA-C16, which was deposited at China General Microbiological Culture Collection Center (CGMCC) on Mar. 28, 2024, with an accession number of CGMCC No. 30175.

Preferably, the host strain is a recombinant HSA-expressing engineered strain constructed by introducing the recombinant expression vector for producing highly-expressed HSA described above into a Pichia pastoris/Komagataella phaffii strain CBS7435.

A use of the host strain for high-level expression of HSA described above in preparation of the HSA is provided.

### 3. Beneficial Effects

Compared with the prior art, the technical solutions provided by the present disclosure have the following beneficial effects:

The present disclosure provides a recombinant expression vector for producing highly-expressed HSA, a host strain, and a use of the host strain. Through the optimization design for the target gene, the construction of a Komagataella phaffii/Pichia pastoris engineered strain carrying the exogenous gene with a high copy number and expressing the exogenous gene at a high level, and the optimization of a pilot-scale fermentation process, an expression level of the recombinant HSA can be as high as 25.82 g/L. A purification process for the recombinant HSA involves only fermentation broth pretreatment and three-step column chromatography, and enables a purity of 99% or more for the target protein. A recovery rate of the entire purification process reaches 60% or more.

### Deposition of Biological Material

The Pichia pastoris HSA-C16 was deposited at the China General Microbiological Culture Collection Center (CGMCC), Institute of Microbiology Chinese Academy of Sciences No. 1 West Beichen Road, Chaoyang District, Beijing, China on Mar. 28, 2024, with an accession number of CGMCC No. 30175.

### BRIEF DESCRIPTION OF THE DRAWINGS

The application file contains at least one drawing executed in color. Copies of this patent or patent application publication with color drawing(s) will be provided by the Office upon request and payment of the necessary fee.

FIG. 1 is a schematic diagram of a computer-simulated alignment curve for the adjustment of a G-C base pair content within a nucleotide sequence shown in SEQ ID NO: 1 for an optimized gene to an optimal level in an embodiment;

FIG. 2 is a map of a Pichia pastoris multi-copy recombinant expression vector pPIC9K in an embodiment;

FIGS. 3A-3B show agarose gel electrophoresis (1% agarose gel) results for the optimized gene in an embodiment;

FIG. 4 is a schematic diagram of a construction process of a recombinant plasmid in an embodiment;

FIG. 5 is a first analysis profile for a nucleotide sequence of the optimized gene in an embodiment, where sequencing results of the nucleotide acid sequence were shown in SEQ ID NO: 6;

FIG. 6 is a second analysis profile for the nucleotide sequence of the optimized gene in an embodiment, where sequencing results of the nucleotide acid sequence were shown in SEQ ID NO: 7;

FIG. 7 is a third analysis profile for the nucleotide sequence of the optimized gene in an embodiment, where sequencing results of the nucleotide acid sequence were shown in SEQ ID NO: 8;

FIG. 8 is a schematic diagram illustrating cell state changes during the electroporation-mediated transformation of the recombinant plasmid into a Pichia pastoris host strain in an embodiment;

FIG. 9 is a schematic diagram illustrating cell state changes during the electroporation-mediated transformation of the recombinant plasmid into a Pichia pastoris host strain GS115 in an embodiment;

FIG. 10 is a schematic diagram illustrating cell state changes during the electroporation-mediated transformation of the recombinant plasmid into a Pichia pastoris host strain CBS7435 in an embodiment;

FIG. 11 shows a first set of results from a small-scale induction experiment for a Pichia pastoris GS115 engineered strain prepared in an embodiment;

FIG. 12 shows a second set of results from a small-scale induction experiment for the Pichia pastoris GS115 engineered strain prepared in the embodiment;

FIG. 13 shows a first set of results from a small-scale induction experiment for a Pichia pastoris CBS7435 engineered strain prepared in an embodiment;

FIG. 14 shows a second set of results from a small-scale induction experiment for the Pichia pastoris CBS7435 engineered strain prepared in the embodiment;

FIG. 15 is a first schematic diagram of protein electrophoresis results at different time points during a 50 L fermenter fermentation test of an engineered strain GS115 (G15) prepared in an embodiment;

FIG. 16 is a second schematic diagram of protein electrophoresis results at different time points during a 50 L fermenter fermentation test of the engineered strain CBS7435 (C7) prepared in the embodiment;

FIG. 17 is a third schematic diagram of protein electrophoresis results at different time points during a 500 L fermenter fermentation test of the engineered strain CBS7435 (C16) prepared in the embodiment;

FIG. 18 shows a standard curve for quantitative determination of a protein concentration with bovine serum albumin as a standard in an embodiment;

FIG. 19 is a schematic diagram of separation and purification results for a supernatant sample from a 50 L fermenter fermentation of an engineered strain prepared in an embodiment;

FIG. 20 shows purity identification data of a human serum albumin sample purified by HPLC analysis in an embodiment;

FIG. 21 is a comparative schematic diagram of non-reduced molecular-weight total ion chromatogram (TIC) analysis between the human serum albumin sample purified in the embodiment and an rHSA national standard;

FIG. 22 is a comparative schematic diagram of reduced molecular-weight TIC analysis between the human serum albumin sample purified in the embodiment and the rHSA national standard; and

FIG. 23 is a comparative schematic diagram of reduced molecular-weight mass spectrometry analysis between the human serum albumin sample purified in the embodiment and the rHSA national standard.

### BRIEF DESCRIPTION OF THE SEQUENCE LISTING

SEQ ID NO: 1 is an artificial DNA sequence of the optimized target gene sequence (i.e. human serum albumin cDNA Length: 1-1755 bp) synthesized in the embodiment;

SEQ ID NO: 2 is an amino acid sequence (Sequence Length: 585aa) corresponding to the optimized target gene sequence of SEQ ID NO: 1;

SEQ ID NO: 3 is a full sequence analytical results of a constructed recombinant plasmid of HSA-pPIC9K in an embodiment;

SEQ ID NO: 4 is an additional 5′ sequence before the optimized target gene sequence inserted two codons in an embodiment;

SEQ ID NO: 5 is an additional 3′ sequence after the optimized target gene sequence inserted two stop codons in an embodiment;

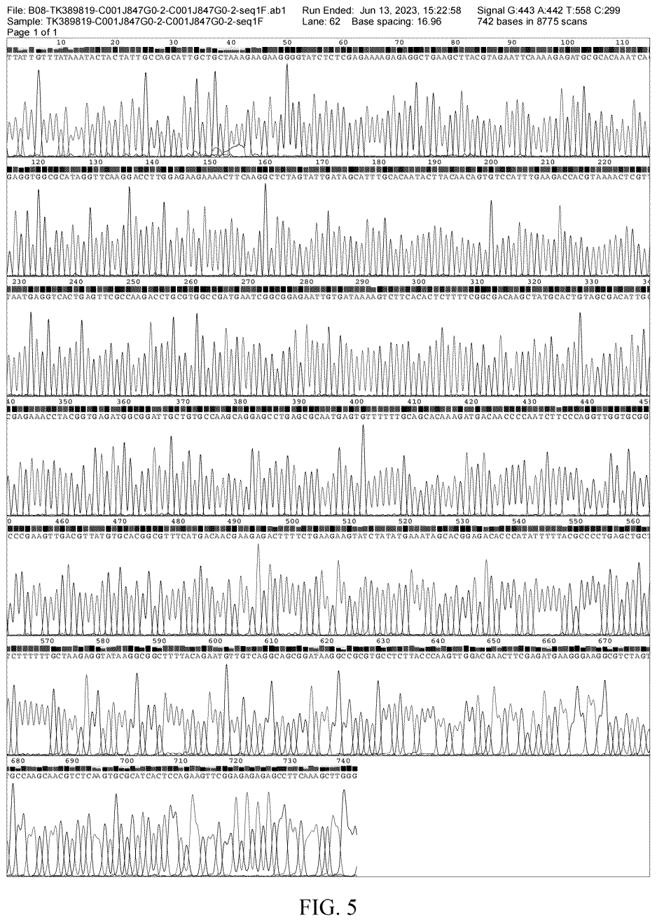

SEQ ID NO: 6 is a sequence alignment analysis of sequencing result (SEQ contig) corresponding to FIG. 5 which is a first analysis profile for a nucleotide sequence of the optimized gene in an embodiment;

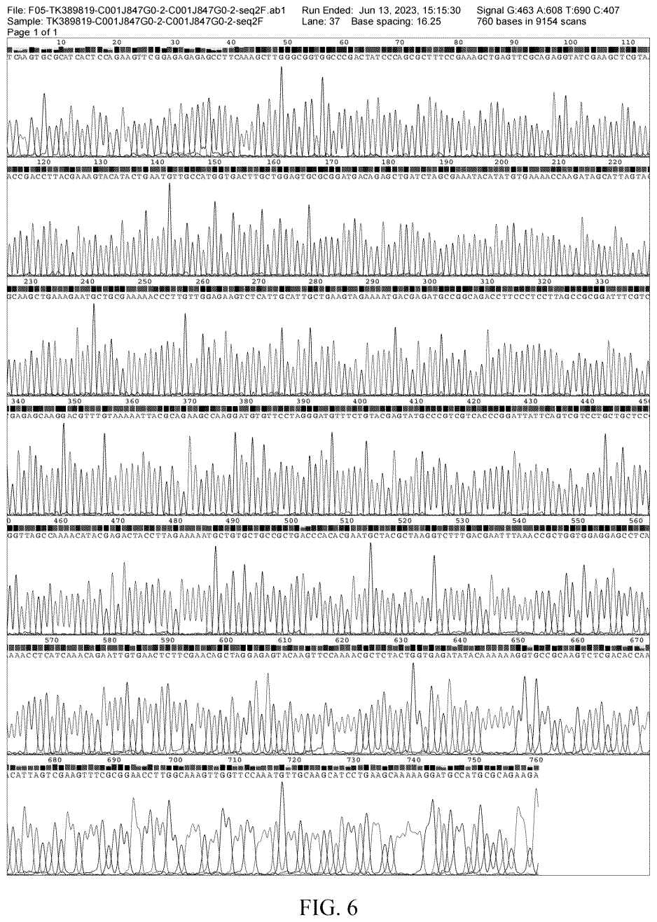

SEQ ID NO: 7 is a sequence alignment analysis of sequencing result (SEQ contig) corresponding to FIG. 6 which is a second analysis profile for a nucleotide sequence of the optimized gene in an embodiment;

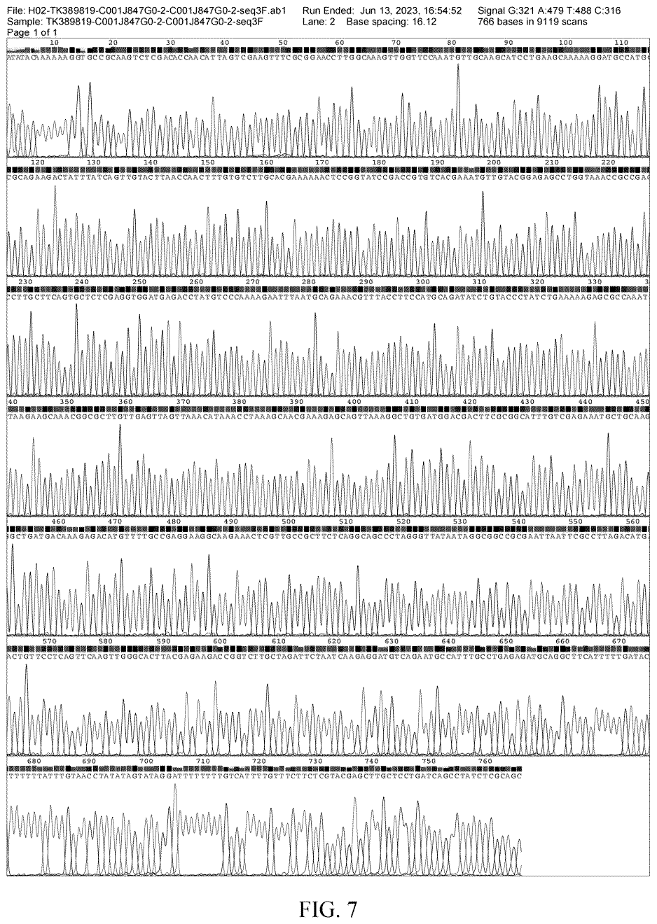

SEQ ID NO: 8 is a sequence alignment analysis of sequencing result (SEQ contig) corresponding to FIG. 7 which is a third analysis profile for a nucleotide sequence of the optimized gene in an embodiment.

### DETAILED DESCRIPTION OF THE EMBODIMENTS

To enable those skilled in the art to well understand the solutions of the present application, the technical solutions in the embodiments of the present application will be clearly and completely described below in conjunction with the accompanying drawings in the embodiments of the present application. Obviously, the described embodiments are only some rather than all of the embodiments of the present application. All other embodiments obtained by those of ordinary skill in the art based on the embodiments of the present application without creative efforts should fall within the protection scope of the present application.

It should be noted that the terms “first”, “second”, etc. In the description and claims of the present application and in the above accompanying drawings are intended to distinguish between similar objects, but do not necessarily indicate a specific order or sequence. It should be understood that data used in such contexts may be interchanged under appropriate circumstances to enable the embodiments of the present application described herein. Moreover, the terms “include”, “has”, and any variants thereof refer to non-exclusive inclusion, for example, a process, method, system, product, or device that includes a series of steps or units is not necessarily limited to those steps or units which are clearly listed, but may include other steps or units which are not expressly listed or inherent to such a process, method, system, product, or device.

In the present application, orientations or positional relationships indicated by terms such as “above”, “below”, “left”, “right”, “front”, “rear”, “top”, “bottom”, “inside”, “outside”, “vertical”, “horizontal”, “transverse”, and “longitudinal” are all based on the orientations or positional relationships illustrated in the accompanying drawings. These terms are mainly intended to well describe the present application and embodiments thereof, rather than to define that devices, elements, or components indicated by these terms must have the specific orientations or must be constructed and operated in the specific orientations.

Besides, some of the terms mentioned above may be used to indicate other meanings in addition to the orientations or positional relationships. For example, the term “upper” may also be used to indicate an attachment relationship or a connection relationship in some cases. Those of ordinary skill in the art may understand specific meanings of these terms in the present application based on a specific situation.

In addition, the terms “mounted”, “arranged”, “provided”, and “connected” should be interpreted in a broad sense. For example, “connection” may be a fixed connection, a removable connection, or integration; may be a mechanical connection or an electrical connection; may be a direct connection or an indirect connection implemented through an intermediate medium; or may be intercommunication between two devices, elements, or components. Those of ordinary skill in the art may understand specific meanings of the above terms in the present application based on a specific situation.

It should be noted that the embodiments in the present application and features in the embodiments may be combined with each other in a non-conflicting situation. The present application will be described in detail below with reference to the accompanying drawings and embodiments.

### Example 1

In this example, a recombinant expression vector for producing highly-expressed HSA is provided. A nucleotide sequence of the recombinant expression vector is shown in SEQ ID NO: 3.

The recombinant expression vector includes:

(1) A 5′ regulatory region that is derived from Pichia pastoris and includes a promoter element. The 5′ regulatory region is selected from a 5′ regulatory region of an AOX1 gene, a DAS1 gene, or a HIS4 gene that is derived from Pichia pastoris. A 3′ terminus of the 5′ regulatory region is linked to a sequence described in (2) below.

(2) An optimized gene encoding the HSA. A nucleotide sequence of the optimized gene is shown in SEQ ID NO: 1. An amino acid sequence of the HSA is shown in SEQ ID NO: 2.

(3) A 3′ termination sequence derived from Pichia pastoris. The 3′ termination sequence is selected from a 3′ termination sequence of the AOX1 gene, an AOX2 gene, or the HIS4 gene that is derived from Pichia pastoris.

The recombinant expression vector further includes:
at least one marker gene for screening in E. coli, such as the ampicillin resistance gene (Amp^{R});
a DNA fragment of a replication origin capable of replicating in an E. coli host strain, such as pBR322 Ori; and
at least two marker genes for screening in a yeast, such as Kan^{R} and HIS4.

The recombinant expression vector is a linearized vector produced by cleaving any one of pPIC9, pPIC3, pPICZαABC, pPIC3.5K, pHIL-S1, pHIL-D2, pA0804, pA0815, pGAPZαABC, pPIC6αABC, and pPIC9K with a restriction endonuclease SacI or BglII.

The recombinant expression vector is a linearized vector produced by cleaving HSA-pPIC9K with the restriction endonuclease SacI or BglII, and the linearized vector includes at least one copy of the optimized gene encoding the HSA.

In the optimized gene encoding the HSA, optimal codons preferred by the AOX1 gene derived from the Pichia pastoris are adopted. A proportion of the optimal codons in total codons of the optimized gene is controlled at 90%.

Three restriction endonuclease sites SalI, HindIII, and XbaI are inserted sequentially in a 5′ to 3′ direction within the optimized gene encoding the HSA, such that the optimized gene is divided into four relatively-balanced large fragments.

In the optimized gene encoding the HSA, consecutive G-C base pairs are reduced and A-T base pairs preferred by the Pichia pastoris are increased, achieving a balanced design of the nucleotide distribution within the optimized gene, such that a GC content in the optimized gene is adjusted to 45% to 50%.

The optimized gene encoding the HSA further includes an expression reading cassette constructed as follows:

A 5′ restriction endonuclease site BamHI is inserted into a 5′ regulatory region (promoter region) of the AOX1 gene. A 10-deoxynucleotide oligonucleotide CCAAACGATG as shown in SEQ ID NO: 9 (including a Kozak sequence for eukaryotic genes, which is AXXATG) is ligated. A yeast α-mating factor leader peptide sequence (including 85 amino acids) derived from Saccharomyces cerevisiae is then ligated. A mature HSA gene is inserted between multiple cloning sites EcoR I and Not I of a pPIC9K recombinant expression vector.

A coding sequence AAAAGA for a dibasic amino acid of lysine and arginine (-Lys-Arg-) is inserted after an enzyme cleavage site EcoR I at a 5′ terminus of the mature HSA gene, where the specific sequence is as shown in SEQ ID NO: 4. A double stop codon TAATAG is inserted before an enzyme cleavage site Not I at a 3′ terminus of the mature HSA gene, where the specific sequence is as shown in SEQ ID NO: 5.

### Example 2

In this example, a host strain for high-level expression of HSA is provided. The host strain is Pichia pastoris HSA-C16, which was deposited at China General Microbiological Culture Collection Center (CGMCC) on Mar. 28, 2024, with an accession number of CGMCC No. 30175.

The host strain is a recombinant HSA-expressing engineered strain constructed by introducing the recombinant expression vector for producing highly-expressed HSA in Example 1 into a Pichia pastoris strain CBS7435.

A use of the host strain in preparation of HSA is provided.

Relevant preparation processes and experimental data for the construction of the recombinant expression vector of Example 1 and the host strain of Example 2 are as follows:

### Materials and Devices:

### 1. Strains

1) Pichia pastoris host strain (GS115 his4 (Mut⁺ his⁻) NRRL Y-15851).

2) Pichia pastoris host strain CBS7435 (the industrial yeast Komagataella phaffii (formerly named Pichia pastoris), named NRRL Y-11430).

3) E. coli host strains:
E. coli JM109 F′(endA1,recA1,gyrA96,thi, sdR17(rₖ⁻,mₖ⁺),relA1,supE44,Δ(lac-proAB), [F′ traD36, proAB, lacI^{q}ZΔM15]; and
E. coli HB101 (supE44 hsd S20(rB-mB−)recA:ara-14 proA₂ lacY:galK₂rpsL20 xyl-5 mtl-1).

The above-mentioned E. coli host strains were used for the cloning of the target gene and the construction and preparation of plasmids.

### 2. Main Reagents

1) DNA restriction endonucleases, T₄ DNA ligase, and polymerases were purchased from GIBCO-BRL, Pharmacia, Bio-Labs, and Cusabio, respectively.

2) Casamino acid (MERK, Germany).

3) Bacto-yeast extract (Difco, USA).

4) Polymerase chain reaction (PCR) amplification kit (Pharmacia, Sweden).

5) DNA sequencing kit (USB, USA).

6) Acrylamide (Acr), N,N-dimethylbisacrylamide (Bis), sodium dodecyl sulfate (SDS), guanidine hydrochloride, urea, and tetramethylethylenediamine (TEMED) (Sigma, UK).

7) Isopropylthio-β-galactoside (IPTG), X-gal, dithiothreitol (DTT), and Agarose (Sigma, UK).

8) YNB, Biotin, and Agar (Difco, USA).

9) Sorbitol, glucose, L-histidine, L-lysine, L-methionine, L-Leucine, L-isoleucine, and L-glutamic acid (Sigma, USA).

10) Glycerol and methanol (Shanghai Chemical Reagent Factory).

11) Enzyme reaction solutions:

Restriction endonuclease high-salt buffer: 10 mM Tris-HCl (pH 7.5), 100 mM NaCl, 10 mM MgCl₂.

Restriction endonuclease medium-salt buffer: 50 mM Tris-HCl (pH 7.5), 50 mM NaCl, 10 mM MgCl₂.

Restriction endonuclease low-salt buffer: 10 mM Tris-HCl (pH 8.0), 10 mM MgCl₂.

T₄ DNA ligase buffer: 50 mM Tris-HCl (pH 8.0), 10 mM MgCl₂, 10 mM DTT, 1 mM ATP.

12) Sodium dodecyl sulfate-polyacrylamide gel electrophoresis (SDS-PAGE) reagents:

Electrophoresis buffer: 192 mM glycine, 25 mM Tris-HCl, and 0.1% SDS, pH 8.3.

Stacking gel buffer: 125 mM Tris-HCl and 0.1% SDS, pH 6.8.

Separating gel buffer: 375 mM Tris-HCl, 0.1% SDS, pH 8.8.

Sample buffer (1×): 50 mM Tris-HCl (pH 6.8), 1% SDS, 10% glycerol, 2.5% mercaptoethanol, and 0.05% bromophenol blue.

30% Acr: 29% Acr, 1% N,N-dimethylbisacrylamide.

Coomassie Brilliant Blue staining solution: 0.25% (W/V) Coomassie Brilliant Blue G-250 and 5% HAc.

45% ethanol destaining solution: 7.5% HAc and 10% ethanol.

13) Common buffers:

TE buffer: 10 mM Tris-HCl (pH 8.0) and 1 mM EDTA.

STE buffer: 10 mM Tris-HCl (pH 8.0), 1 mM EDTA, and 20 mM NaCl.

Phosphate-buffered saline (PBS): 10 mM NaH, P0q-Na, HPO₃ (pH 7.0), and 150 mM NaCl.

10×TBS buffer: per liter: 108 g of Tris base, 55 g of boric acid, and 40 mL of EDTA (0.5 M), pH 8.0.

50×TAE buffer: per liter: 242 g of Tris base, 57.1 mL of glacial acetic acid, and 100 mL of EDTA (0.5 M), pH 8.0.

Saturated phenol: Commercially-available phenol was redistilled, saturated with TE buffer, dispensed, and stored at −20° C.

Phenol:chloroform:isoamylalcohol (V/V): 1:1:0.8.

Chloroform:isoamylalcohol (V/V): 24:1.

### 3. Main Instruments and Equipment

1) ABI381A DNA automatic synthesizer, Applied Biosystems (ABI), USA.

2) 5 L RIBE-5 automatic fermenter, Shanghai Guoqiang Biochemical Engineering Equipment Co., Ltd.

3) 15 L Biocenter 15F automatic fermenter, Shanghai Guoqiang Biochemical Engineering Equipment Co., Ltd. (Software system: Fermentation Star).

4) 50 L Biocenter 50F automatic fermenter, Shanghai Guoqiang Biochemical Engineering Equipment Co., Ltd. (Software system: Fermentation Star).

5) 100 L Biocenter 100F automatic fermenter, Shanghai Guoqiang Biochemical Engineering Equipment Co., Ltd. (Software system: Fermentation Star).

6) 500 L automatic BIOSTAT® Bplus bioreactor, B. Braun, Germany.

7) SDL-100 protein purification system, Suzhou SePure Instruments Co., Ltd. (medium-pressure chromatography workstation, software SCG).

8) Agilent High-Performance Liquid Chromatograph, 1260 Infinity, Agilent Technologies, USA.

9) External-pressure ultrafiltration membrane module (filtration accuracy: 30 KD), Huzhou MFL Membrane Technology Co., Ltd.

10) External-pressure ultrafiltration membrane module (filtration accuracy: 100 KD), Huzhou MFL Membrane Technology Co., Ltd.

11) Full-wavelength microplate reader, Thermo Fisher 1510, Thermo Fisher Scientific, USA.

12) Electroporation instrument, Micropulser 411BR10654, BIO-RAD, USA.

### 4. Experimental Methods

### (1) Media and Culture Conditions

E. coli liquid medium LB (1% of bacto-tryptone, 0.5% of bacto-yeast extract, and 1% of NaCl). A corresponding solid medium was produced by adding an agar powder at 15 g/L to the liquid medium. E. coli was cultured at 37° C. Pichia pastoris rich medium YPD (1% of yeast extract, 2% of peptone, and 2% of dextrose). Pichia pastoris protoplast regeneration medium RDB [1 M of sorbitol, 1% of dextrose, 1.34% of YNB, 4×10⁵ of biotin, 0.005% of an amino acid mixture (including L-glutamic acid, L-methionine, L-leucine, L-isoleucine, and L-lysine)]. A corresponding solid medium was produced by adding an agar powder at 2% to the liquid medium. Pichia pastoris shake flask media BMGY and BMMY (1% of yeast extract, 2% of peptone, 100 mM of PBS, pH 6.0, 1.34% of YNB, 4×10⁵% of biotin, 1% of glycerol, or 0.5% of methanol). Pichia pastoris was cultured at 30° C.

(2) First High-Density Fermentation Medium and Culture Conditions for Pichia pastoris in a Fermenter:

| A. 10 × Basal Salts: | | |
|---|---|---|
| 1. H₃PO₄, 85% | 42 | ml |
| 2. CaSO₂•2H₂O | 1.8 | g/L |
| 3. K₂SO₄ | 28.6 | g/L |
| 4. MgSO₄•7H₂O | 23.4 | g/L |
| 5. KOH | 6.5 | g/L |
| B. 250 × PTMl salts: | | |
| 1. CuSO₄•5H₂O | 6 | g/L |
| 2. KI | 0.08 | g/L |
| 3. MnSO₄•H₂O | 3 | g/L |
| 4. Na₂MoO₄•2H₂O | 0.2 | g/L |
| 5. H₃BO₃ | 0.02 | g/L |
| 6. CoCl₂ | 0.5 | g/L |
| 7. ZnCl₂ | 20 | g/L |
| 8. FeSO₄•7H₂O | 65 | g/L |
| 9. Biotin | 0.2 | g/L |
| 10. H₂SO₄ | 5 | mL |
| C. glycerol | 8% | (V/V) |
| D. Feed medium | 50% glycerol(1 L) + 12 mL/L PTM₁ | |
| E. Induced medium | 100% methanol (1 L) + 12 mL/L PTM₁ | |
| F. Dissolved O₂ (DO) | >20% | |
| G. pH | 5.0-5.8 | |
| H. Temperature | 20-30° | C. |

(3) Second High-Density Fermentation Medium and Culture Conditions for Pichia pastoris in a Fermenter:

### Preparation of a Solution A:

YPD medium (50 mL):

| | |
|---|---|
| 1. Yeast extract | 0.5 g |
| 2. peptone | 1 g |
| 3. Glucose | 1 g |

The YPD medium was used for culturing a primary seed culture of Pichia pastoris.

### Preparation of a Solution B:

BSM medium (1 L):

| | | |
|---|---|---|
| 1. 98% glycerol | 63 | g/L |
| 2. ddH₂O (Ultrapure water) | 912 | mL |
| 3. CaSO₂•2H₂O | 0.46 | g/L |
| 4. MgSO₄•7H₂O | 5.84 | g/L |
| 5. K₂SO₄ | 7.34 | g/L |
| 6. (NH₄)₂SO₄ | 9 | g/L |

The BSM medium was used for culturing a secondary seed culture of Pichia pastoris and served as a primary nutrient medium for yeast culture in a bioreactor.

### Preparation of a Solution C:

Preparation of a sodium hexametaphosphate (HMP) solution (150 mL):

| | |
|---|---|
| 1) (NaPO₃)₆ (Sodium hexametaphosphate) | 30 g |
| 2) ddH₂O (ultrapure water) | 150 mL |

### Preparation of a Solution D:

PTM₁ solution (1 L):

| | | |
|---|---|---|
| 1. CuSO₄•5H₂O | 6 | g/L |
| 2. KI | 0.08 | g/L |
| 3. MnSO₄•H₂O | 3 | g/L |
| 4. Na₂MoO₄•2H₂O | 0.2 | g/L |
| 5. H₃BO₃ | 0.02 | g/L |
| 6. CoCl₂ | 0.5 | g/L |
| 7. ZnCl₂ | 20 | g/L |
| 8. FeSO₄•7H₂O | 65 | g/L |
| 9. Biotin | 0.2 | g/L |
| 10. H₂SO₄ | 5 | mL |

### (4) Plasmid Extraction

### 1) Small-Scale Plasmid Extraction

Single colonies were picked and inoculated into 2 mL of an LB medium including a corresponding antibiotic, and cultured overnight at 37° C. under shaking. The next day, 1.5 mL of a resulting culture was collected in an Eppendorf tube and centrifuged to produce a cell pellet and a supernatant, and the supernatant was discarded. The cell pellet was placed in an ice bath. 100 μL of a solution I (50 mM glucose, 25 mM Tris-HCl, and 10 mM EDTA, pH 8.0) was added, shaking was conducted for thorough mixing, and incubation was conducted at room temperature for 5 min. 200 μL of a solution II (0.2 N NaOH and 1% SDS) was added, inverting was repeated for thorough mixing, and incubation was conducted in an ice bath for 5 min. 150 μL of a solution III (3 M NaAc, pH 4.8) was added, gentle shaking was conducted for thorough mixing, and incubation was conducted in an ice bath for 5 min. 450 μL of redistilled phenol/chloroform (1:1) was added, and thorough mixing was conducted. Centrifugation was conducted at 12,000 rpm for 10 min. A resulting upper aqueous phase was carefully transferred to pre-cooled absolute ethanol in a volume 2 times the volume of the upper aqueous phase, and a resulting mixture was placed in a −20° C. refrigerator for 2 h and centrifuged at 12,000 rpm for 15 min. A resulting ethanol phase was removed, and 500 μL of 70% ethanol was carefully added for washing to remove salts. Centrifugation and vacuum-drying were conducted. 18 μL of TE and 2 μL of an RNase solution were added, and incubation was conducted at 37° C. for 1 h. A resulting sample was for enzyme digestion analysis and further cloning.

### 2) Large-Scale Plasmid Extraction

500 mL of a strain solution produced after culturing overnight was collected and centrifuged at 5,000 rpm and 4° C. for 10 min. Washing was conducted with 100 mL of STE. Then, centrifugation was conducted, and a resulting cell pellet was collected. 18 mL of a solution I (50 mM glucose, 25 mM Tris-HCl, and 10 mM EDTA, pH 8.0) and 2 mL of a lysozyme (10 mg/mL, 1% SDS) were added successively, and incubation was conducted at room temperature for 10 min. 40 mL of a solution II (0.2 N NaOH and 1% SDS) was added, thorough mixing was conducted gently (until a resulting solution was transparent), and incubation was conducted in an ice bath for 5 min. Then 20 mL of a solution III (3 M NaAc, pH 4.8) was added, thorough mixing was conducted, and incubation was conducted in an ice bath for 10 min. Extraction was conducted twice with a phenol-chloroform (volume ratio: 1:1) solution. Pre-cooled absolute ethanol was added in a two-fold volume, and incubation was conducted overnight at −20° C. Centrifugation was conducted at 12,000 rpm for 15 min, and a resulting supernatant was discarded. A resulting precipitate was washed 1 time to 2 times with 70% ethanol, then vacuum-dried, and dissolved in 3 mL of TE. 10 μL of an RNase solution (10 mg/mL) was added, and incubation was conducted at 37° C. for 30 min. A resulting sample was loaded onto a Sepharose 2B chromatographic column (1×10 cm, pre-equilibrated with TE). A first peak (high-molecular-weight DNA) was collected with TE (pH 7.6) as an eluent. The volume of a collected product was measured. Pre-cooled absolute ethanol in a volume 2 times the volume of the collected product and 3 M NaAc (pH 5.2) in a volume 1/10 of the volume of the collected product were added. A resulting mixture was thoroughly mixed, placed overnight in a −20° C. refrigerator, and centrifuged at 12,000 rpm for 15 min. A resulting supernatant was discarded. A resulting precipitate was washed 1 time to 2 times with 70% ethanol, vacuum-dried, and dissolved in 1 mL of TE. An appropriate amount of the resulting sample solution was taken and tested for a DNA concentration using an ultraviolet spectrophotometer at 260 nm. Recombinant plasmid DNA obtained from the large-scale extraction could be used for the protoplast-mediated transformation into a Pichia pastoris host strain and the long-term cryopreservation in the present disclosure.

(5) Preparation of E. coli Competent Cells and Transformation of the Recombinant Plasmid

An E. coli host strain was inoculated into 2 mL of an LB medium and cultured overnight at 37° C. under shaking. The next day, 500 μL of a resulting culture was inoculated into 50 mL of an LB medium and cultured for 1 h at 37° C. and 300 rpm under shaking until a cell density reached OD₆₀₀=0.5. Centrifugation was conducted for 5 min at 5,000 rpm and 4° C. A resulting supernatant was decanted. A resulting cell pellet was resuspended in 25 mL of a cold solution including 100 mM of CaCl₂ and 10 mM of Tris-HCl (pH 7.4), placed in an ice bath for 30 min to 60 min, and then centrifuged for 5 min at 5,000 rpm and 4° C. A resulting supernatant was decanted. A resulting cell pellet was resuspended in 2 mL of a cold calcium chloride solution, placed in an ice bath for 40 min, and then could be used for transformation.

200 μL of the freshly-prepared E. coli competent cell suspension was added to an Eppendorf tube, and 10 μL of a recombinant plasmid DNA-containing ligation solution were added. Incubation was conducted in an ice bath for 30 min, and a heat shock was conducted at 42° C. for 2 min. 500 μL of an LB medium was added, and incubation was conducted for 1 h on a shaker at 37° C. under gentle shaking. Centrifugation was conducted at 10,000 rpm for 10 s. Most of the resulting LB medium supernatant was discarded with approximately 200 μL left. A resulting bacterial suspension was thoroughly mixed gently with a pipette tip, and then divided into two portions of 50 μL and 150 μL. Each portion was coated on an LB agarose plate including 50 μg/mL of ampicillin, and cultured in a 37° C. incubator for 8 h to 16 h.

In this example, a method for efficiently producing high-purity recombinant HSA is provided. The method includes the following steps:

S1. A host strain was constructed. The host strain was deposited at China General Microbiological Culture Collection Center (CGMCC) on Mar. 28, 2024, with an accession number of CGMCC No. 30175.

S2. Fermentation.

S3. Purification.

The host strain was a recombinant HSA-expressing engineered strain constructed by introducing the recombinant expression vector into a Pichia pastoris strain CBS7435. A nucleotide sequence of the recombinant expression vector was shown in SEQ ID NO: 3.

The fermentation in the S2 specifically includes:

S210. Medium preparation.

S220. Electrode calibration.

S230. Feeding.

S240. Inoculation: The host strain highly expressing the HSA was inoculated.

S250. Fermentation.

S260. Harvesting.

The recombinant expression vector includes:

1. A 5′ regulatory region that is derived from Pichia pastoris and includes a promoter element. The 5′ regulatory region is selected from a 5′ regulatory region of an AOX1 gene, a DAS1 gene, or a HIS4 gene that is derived from Pichia pastoris. A 3′ terminus of the 5′ regulatory region is linked to a sequence described in (2) below.

2. An optimized gene encoding the HSA. A nucleotide sequence of the optimized gene is shown in SEQ ID NO: 1. An amino acid sequence of the HSA is shown in SEQ ID NO: 2.

3. A 3′ termination sequence derived from Pichia pastoris. The 3′ termination sequence is selected from a 3′ termination sequence of the AOX1 gene, an AOX2 gene, or the HIS4 gene that is derived from Pichia pastoris.

For the optimized gene encoding the HSA, a nucleotide sequence has a length of 1,755 bp, and an amino acid sequence encoded correspondingly includes 585 amino acids. The optimized gene is one of the longest functional genes fully chemically synthesized internationally to date.

The present disclosure relates to a specific and novel optimization design for the HSA gene. The design is based on the following three core molecular biology databases: 1. International Nucleotide Sequence Database (GenBank/EMBL/DDBJ). 2. Swiss Protein Sequence and Annotation Database (Swiss-PROT). 3. Protein and Biomolecular Three-Dimensional Structure Database (Protein Data Bank, PDB) provided by the U.S. Brookhaven National Laboratory. The comprehensive auxiliary analysis was conducted using various computer software packages (including the GENESIS and PROSIS packages from the Genetic Computer Group of the University of Wisconsin, the Caltec package from the California Institute of Technology, the DNASIS and PROSIS packages provided by Pharmacia in Sweden, and other programs) to ultimately produce the optimized gene encoding the HSA.

The recombinant expression vector further includes:

At least one marker gene for screening in E. coli, such as the ampicillin resistance gene (Amp^{R}).

A DNA fragment of a replication origin capable of replicating in an E. coli host strain, such as pBR322 Ori.

At least two marker genes for screening in a yeast, such as Kan^{R} and HIS4.

In the optimized gene encoding the HSA, optimal codons preferred by the AOX1 gene derived from the Pichia pastoris are adopted. A proportion of the optimal codons in total codons of the optimized gene is controlled at 90%.

Three restriction endonuclease sites SalI, HindIII, and XbaI are inserted sequentially in a 5′ to 3′ direction within the optimized gene encoding the HSA, such that the optimized gene is divided into four relatively-balanced large fragments, which facilitates the splicing, cloning, and assembly of the synthesized gene.

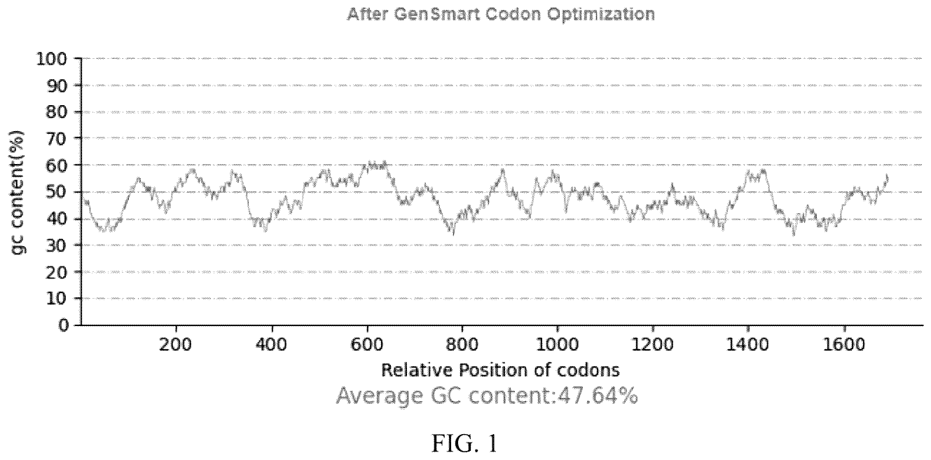

In the optimized gene encoding the HSA, consecutive G-C base pairs are reduced and A-T base pairs preferred by Pichia pastoris are increased, such that a GC content in the optimized gene is adjusted to 45% to 50%. In this example, the optimal GC content is preferably 47.64%, which facilitates the high-level expression of the target gene (as shown in FIG. 1).

The optimized gene encoding the HSA further includes an expression reading cassette constructed as follows:

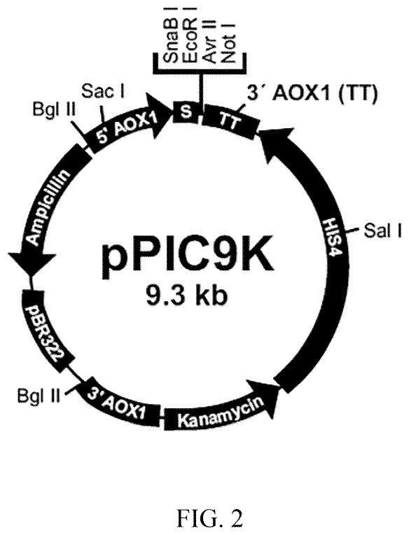

A 5′ restriction endonuclease site BamHI was inserted into a 5′ regulatory region (promoter region) of the AOX1 gene. A 10-deoxynucleotide oligonucleotide CCAAACGATG as shown in SEQ ID NO: 9 (including a Kozak sequence for eukaryotic genes, which was AXXATG) was ligated. A yeast α-mating factor leader peptide sequence (including 85 amino acids) derived from Saccharomyces cerevisiae was then ligated. A mature HSA gene was inserted between multiple cloning sites EcoRI and NotI of a pPIC9K recombinant expression vector (as shown in FIG. 2).

A coding sequence AAAAGA for a dibasic amino acid of lysine and arginine (-Lys-Arg-) was inserted after an enzyme cleavage site EcoRI at a 5′ terminus of the mature HSA gene, where the specific sequence is as shown in SEQ ID NO: 4. A double stop codon TAATAG was inserted before an enzyme cleavage site NotI at a 3′ terminus of the mature HSA gene, where the specific sequence is as shown in SEQ ID NO: 5. As shown in FIG. 2, the coding sequence AAAAGA for the dibasic amino acid of lysine and arginine (-Lys-Arg-) was inserted after the enzyme cleavage site EcoRI at the 5′ terminus of the target gene to facilitate the processing and cleavage by an endogenous KEX-2 protease in yeast. The double stop codon TAATAG was inserted before the enzyme cleavage site NotI at the 3′ terminus of the target gene to enhance a translation termination signal and prevent read-through during gene expression.

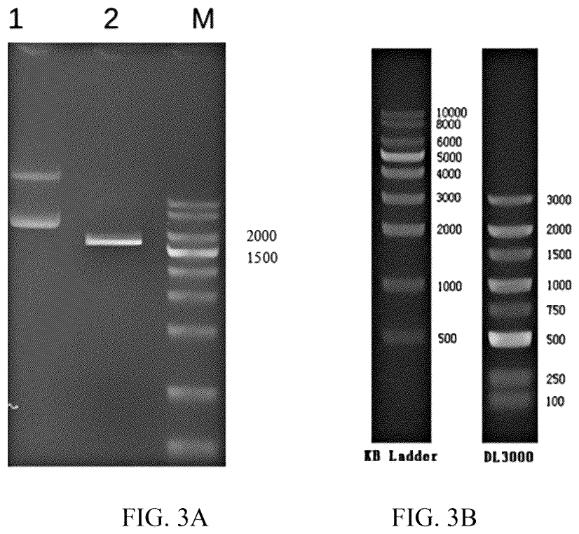

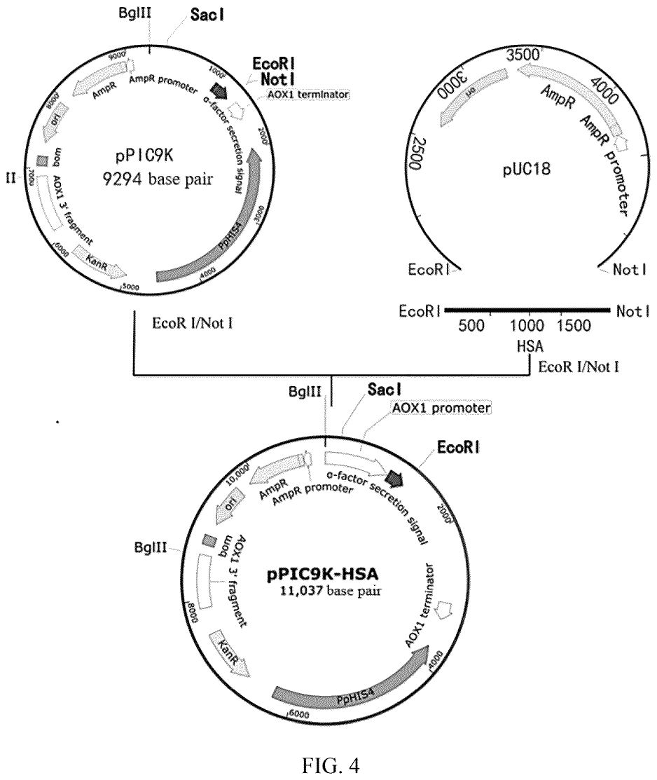

A fully chemically synthesized mature gene (1,755 bp) encoding the HSA was excised from a pUC18 cloning vector through EcoRI-Smal double enzyme digestion. Resulting fragments were analyzed by 1% agarose gel electrophoresis. Results are shown in FIGS. 3A-3B (FIG. 3A: Lane 1 represents the recombinant plasmid, lane 2 represents a recombinant plasmid digested with EcoRI and Smal, and lane M represents a molecular weight standard KB Ladder. FIG. 3B: Two lanes represent molecular weight standards KB Ladder and DL3000, respectively), which are consistent with the expected results. A multiple cloning site KpnI in the pUC18 cloning vector was replaced with NotI. The fully chemically synthesized mature gene (1,755 bp) encoding the HSA was excised from the pUC18 cloning vector through EcoRI-NotI double enzyme digestion. A gel band with an expected size was cut, and a target gene fragment was recovered with a DNA recovery kit. The HSA fragment excised from the pUC18 cloning vector through the EcoRI-NotI double enzyme digestion was cloned into a Pichia pastoris recombinant expression vector pPIC9K undergoing the corresponding double enzyme digestion to construct a recombinant expression vector HSA-pPIC9K (carrying an HSA EcoRI-NotI fragment). A flow chart of the specific construction of the recombinant vector is shown in FIG. 4. A ligation reaction system is as follows:

| | |
|---|---|
| HSA DNA fragment (60 ng) | 4 μL |
| T₄ DNA ligase (2 U) | 1 μL |
| pPIC9K vector (240 ng) | 5 μL |
| ddH₂0 | 6 μL |
| 5 x ligase buffer | 4 μL. |

Ligation was conducted overnight in a 20 μL reaction system at 16° C.

4 μL and 8 μL of the above ligation solution including the recombinant plasmid HSA-pPIC9K were used to transform competent E. coli JM109 host cells. Transformants were coated on LB plates (including 20 μg/mL of ampicillin), and cultured overnight in a 37° C. incubator. 18 colonies were randomly selected from transformed colonies carrying the recombinant plasmid HSA-pPIC9K, inoculated into 2 mL of an LB medium (including 20 μg/mL of ampicillin), and cultured for 6 h to 8 h on a shaker under shaking. Plasmid double-stranded DNA was rapidly extracted using an alkaline denaturation method, and then subjected to EcoRI-NotI double enzyme digestion. 1% agarose gel electrophoresis was conducted to identify recombinants carrying the HSA gene of the expected size. Five clones carrying the HSA EcoRI-NotI fragment were identified. The identified E. coli host strain carrying the HSA-pPIC9K recombinant plasmid was inoculated and cultured. A large amount of the HSA-pPIC9K recombinant plasmid was extracted through large-scale plasmid extraction for long-term storage or for subsequent transformation into a Pichia pastoris host strain. The specific design details and sequencing results for the construction of the HSA-pPIC9K recombinant plasmid are shown in FIG. 4, FIG. 5, FIG. 6, and FIG. 7. The sequence alignment analysis of sequencing result (SEQ contig) are exhibited in SEQ ID NO: 6, SEQ ID NO: 7 and SEQ ID NO: 8.

The recombinant plasmid was linearized with a restriction endonuclease SacI or BglII. A linearized recombinant plasmid was then transformed into a Pichia pastoris host strain GS115 or CBS7435 through electroporation. A specific implementation process was as follows:

a. Preparation of Media

1. BMGY medium: 10 g of yeast extract and 20 g of peptone were dissolved in 700 mL of deionized water, subjected to moist-heat sterilization for 20 min, and cooled. Then, 100 mL of 1 M potassium phosphate buffer at pH 6.0, 100 mL of 10× yeast nitrogen base (YNB), 2 mL of 500×B, and 100 mL of 10× GY) were added. The BMGY medium was stored at 4° C.

2. BMMY medium: 10 g of yeast extract and 20 g of peptone were dissolved in 700 mL of deionized water, subjected to moist-heat sterilization for 30 min, and cooled. Then, 100 mL of 1 M potassium phosphate buffer at pH 6.0, 100 mL of 10×YNB, 2 mL of 500×B, and 100 mL of 10× M were added. The BMMY medium was stored at 4° C.

3. MD medium: 100 mL of 10×YNB, 2 mL of 500×B, and 100 mL of 10× D were added to 800 mL of sterile water. The MD medium was stored at 4° C.

4. 1 M potassium phosphate buffer with pH 6.0:132 mL of 1 M K₂HPO₄ and 868 mL of 1 M KH₂PO₄ were mixed, adjusted to a pH of 6.0, subjected to moist-heat sterilization, and stored at 4° C.

5. 10×YNB: 134 g of YNB (including ammonium sulfate) was dissolved in 1,000 mL of deionized water, and a resulting solution was filtered for sterilization and stored at 4° C.

6. 500×B: 20 mg of biotin was dissolved in 100 mL of deionized water, and a resulting solution was filtered for sterilization and stored at 4° C.

7. 10× M: 5 mL of methanol and 95 mL of deionized water were mixed, filtered for sterilization, and stored at 4° C.

8. 10× GY: 100 mL of glycerol and 900 mL of deionized water were mixed, subjected to moist-heat sterilization, and then stored at 4° C.

9. 10× D: 100 g of glucose was dissolved in 1,000 mL of deionized water, and a resulting solution was filtered for sterilization and stored at 4° C.

10. 1 M sorbitol: 18.2 g of D-sorbitol was dissolved in 100 mL of deionized water, and a resulting solution was filtered for sterilization and stored at 4° C.

b. Preparation of Microbial Cells

1. Single colonies of Pichia pastoris (GS115 or CBS7435) were picked, inoculated into a 50 mL Erlenmeyer flask with 5 mL of an YPD medium, and cultured overnight at 30° C. and 250 r/min to 300 r/min.

2. 100 μL to 500 μL of a resulting culture was inoculated into a 2 L Erlenmeyer flask with 500 mL of a fresh medium, and cultured overnight at 28° C. to 30° C. and 250 r/min to 300 r/min until OD₆₀₀ reached 1.3 to 1.5.

3. A resulting cell culture was centrifuged at 4° C. and 1,500×g for 5 min. A resulting cell pellet was resuspended in 500 mL of ice-cooled sterile water.

4. Centrifugation was conducted according to step 3, and a resulting cell pellet was resuspended in 250 mL of ice-cooled sterile water.

5. Centrifugation was conducted according to step 3, and a resulting cell pellet was resuspended in 20 mL of an ice-cooled 1 M sorbitol solution.

6. Centrifugation was conducted according to step 3, and a resulting cell pellet was resuspended in 1 mL of an ice-cooled 1 M sorbitol solution with a final volume of approximately 1.5 mL.

Note: A resulting cell suspension can be dispensed in 80 μL portions and frozen, but the transformation efficiency will be compromised accordingly (within 2 weeks).

c. Electroporation

1. 80 μL of the prepared Pichia pastoris competent cell and 5 μg to 20 μg of the linearized DNA (dissolved in 5 μL to 10 μL of double-distilled water) were added to a pre-cooled 1.5 mL centrifuge tube, thoroughly mixed, and then transferred to an electroporation cuvette (0.2 cm gap) pre-treated in an ice bath.

2. The electroporation cuvette with the transformation mixture was incubated in an ice bath for 5 min.

3. According to specifications for an electroporation instrument and with reference to other references and repeated explorations, appropriate parameters such as a voltage, a current, and a capacitance were determined. Electroporation was conducted under the optimized parameters. The preferred parameters recommended in the present disclosure were as follows: voltage: 1,500 V to 1,800 V, capacitance: 25 μF, resistance: 200Ω to 400Ω, and electric shock duration: 4 msec to 10 msec.

4. Immediately after a pulse, 1 mL of an ice-cooled 1 M sorbitol solution was added to the electroporation cuvette, and a resulting transformation system was then transferred to a new 1.5 mL centrifuge tube.

5. Incubation was conducted for 1 h to 2 h on a shaker at 28° C.

6. 50 μL to 200 μL of a transformed Pichia pastoris host strain GS115 or CBS7435 solution was coated on MD plates.

7. Culturing was conducted in a 28° C. incubator for approximately 3 d to 4 d until numerous monoclonal positive colonies carrying the recombinant plasmid (yeast transformants) grew.

d. Screening of Multi-Copy Transformants

1. An MD plate on which yeast transformants grew was fully rinsed with 2 mL of sterile water, and a resulting transformant solution was drawn by a pipette and temporarily stored in a centrifuge tube.

2. The transformant solution was coated on YPD plates with G418 resistance, and 100 μL of the transformant solution was coated on each plate. G418 antibiotic contents in the YPD plates were set to 0.25%, 0.5%, 1.0%, 1.25%, 1.5%, 1.75%, 2.0%, etc., respectively.

3. Plates coated with the transformant solution were incubated in a 28° C. incubator for 4 d to 12 d until white monoclonal colonies with varying G418 resistance levels grew (that is, Pichia pastoris transformants with different copy numbers were acquired).

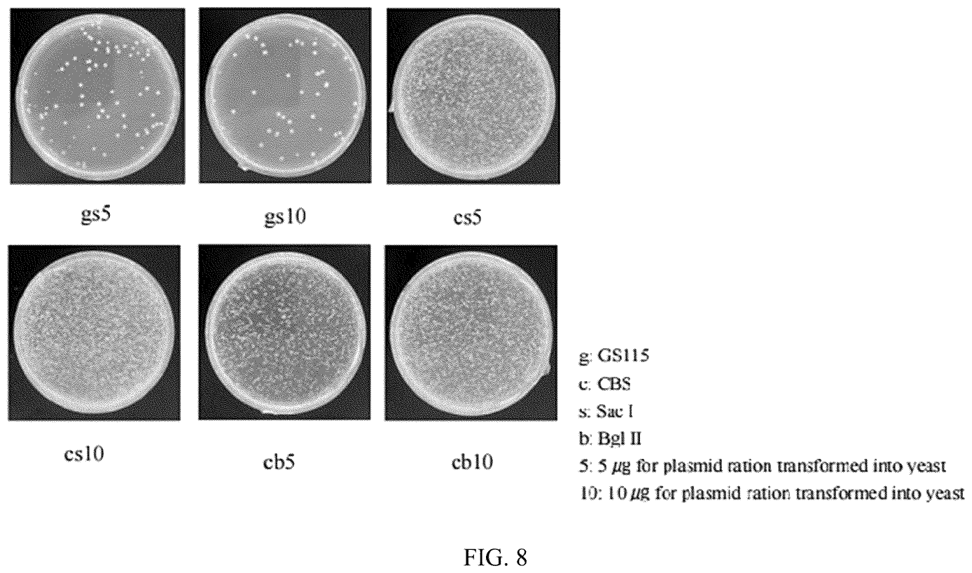

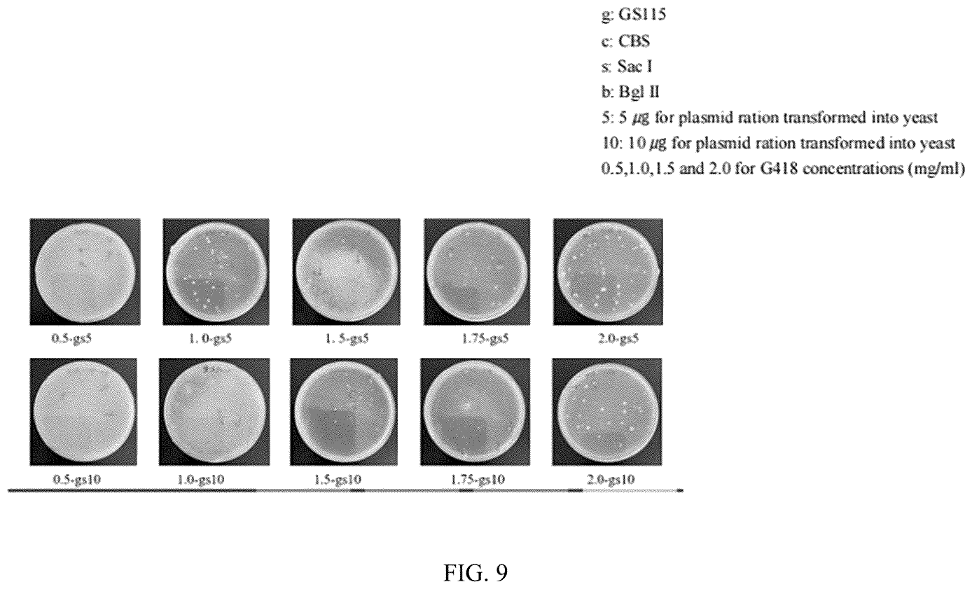

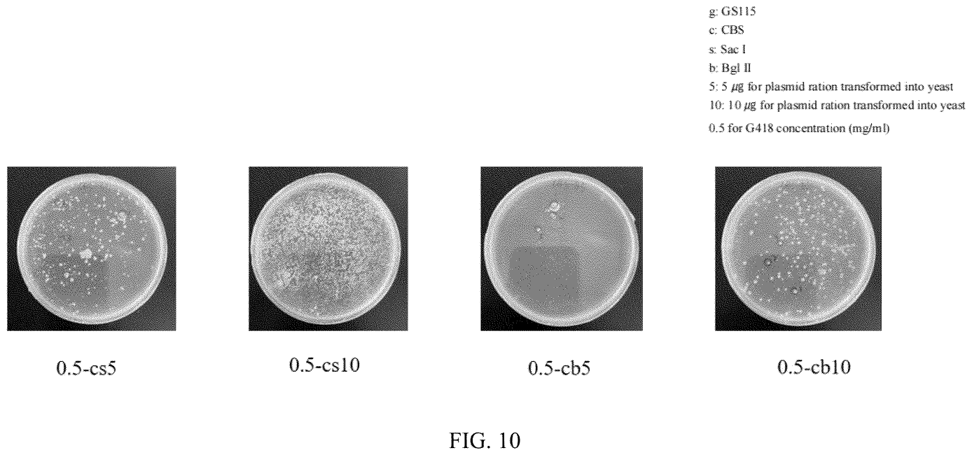

Experimental results of the electroporation transformation of the host strain GS115 or CBS7435 in this example are partially shown in FIG. 8, FIG. 9, FIG. 10, and Tables 1 and 2.

TABLE 1
Experimental results of multi-copy Pichia pastoris
GS115 engineered strains

| | | | Linearization | Plasmid for |
|---|---|---|---|---|
| | Competent cell | G418 | enzyme | electroporation |
| No. | identifier | resistance | SacI/BglII | (μg) |
| G1 | g | 0.50 | S | 5 |
| G2 | g | 0.50 | S | 5 |
| G3 | g | 0.50 | S | 5 |
| G4 | g | 0.50 | S | 10 |
| G5 | g | 0.50 | S | 10 |
| G6 | g | 0.50 | S | 10 |
| G7 | g | 1.00 | S | 5 |
| G8 | g | 1.00 | S | 5 |
| G9 | g | 1.00 | S | 5 |
| G10 | g | 1.00 | S | 10 |
| G11 | g | 1.00 | S | 10 |
| G12 | g | 1.00 | S | 10 |
| G13 | g | 1.75 | S | 5 |
| G14 | g | 1.75 | S | 5 |
| G15 | g | 1.75 | S | 5 |
| G16 | g | 1.75 | S | 10 |
| G17 | g | 1.75 | S | 10 |
| G18 | g | 1.75 | S | 10 |
| G19 | g | 1.50 | S | 5 |
| G20 | g | 1.50 | S | 5 |
| G21 | g | 1.50 | S | 5 |
| G22 | g | 1.50 | S | 10 |
| G23 | g | 1.50 | S | 10 |
| G24 | g | 1.50 | S | 10 |

Note:
In Table 1, G418 resistance of 0.25 indicates that a host strain carries one copy of the exogenous gene, and so on.

TABLE 2
Experimental results of screening of multi-copy
Pichia pastoris CBS7435 engineered strains

| | Small-scale | Competent | | Linearization | Plasmid for |
|---|---|---|---|---|---|
| | induction | cell | G418 | enzyme | electropora- |
| No. | batch | identifier | resistance | SacI/BglII | tion (μg) |
| C1 | 1 | c | 1.0 | S | 5 |
| C2 | 2 | c | 0.25 | S | 5 |
| C3 | 2 | c | 0.25 | S | 5 |
| C4 | 2 | c | 0.5 | S | 5 |
| C5 | 2 | c | 0.5 | S | 5 |
| C6 | 2 | c | 0.5 | S | 5 |
| C7 | 2 | c | 1.0 | S | 5 |
| C8 | 2 | c | 1.0 | S | 5 |
| C9 | 2 | c | 1.0 | S | 5 |
| C10 | 3 | c | 1.0 | S | 5 |
| C11 | 3 | c | 1.0 | S | 5 |
| C12 | 4 | c | 1.25 | S | 5 |
| C13 | 4 | c | 1.25 | S | 5 |
| C14 | 4 | c | 1.25 | S | 5 |
| C15 | 4 | c | 1.5 | S | 5 |
| C16 | 4 | c | 1.75 | S | 5 |
| C17 | 4 | c | 1.75 | S | 5 |
| C18 | 4 | c | 1.75 | S | 5 |
| C19 | 5 | c | 0.5 | S | 5 |
| C20 | 5 | c | 0.5 | S | 10 |
| C21 | 5 | c | 0.5 | S | 10 |
| C22 | 5 | c | 0.5 | S | 10 |

Note:
In Table 2, G418 resistance of 0.25 indicates that a host strain carries one copy of the exogenous gene, and so on.

For each of the transformants carrying the recombinant plasmid HSA-pPIC9K carrying the exogenous gene with different copy numbers selected by the electroporation method, six single colonies were randomly picked from an YPD plate, inoculated into a test tube with 4 mL of a BMGY liquid medium, and cultured overnight under shaking at 28° C. and 200 rpm to 250 rpm. A resulting culture in the test tube was centrifuged for 5 min at 1,500 g to 3,000 g and room temperature. A resulting cell pellet was collected and resuspended in 4 mL (equivalent to the original culture volume) of a BMMY medium and cultured for 36 h to 48 h under shaking at 28° C. and 250 rpm to 300 rpm. Centrifugation was conducted at 5,000 rpm for 10 min. 30 μL of a resulting supernatant was collected and vacuum-dried, and then tested by 10% SDS-PAGE. High-expression clones were identified with an expression product of a pPIC9K empty vector as a control. The selected high-expression clones were then preserved in 15% glycerol at a low temperature as an engineered strain for further small-scale induction expression experiments.

If the high-level-expression engineered strain was a Pichia pastoris GS115 strain, a small-scale induction expression experiment was conducted according to the following experimental protocol: glycerol stock→YPD→BMGY→BMGY→BMMY. Specifically, the glycerol stock was inoculated into a YPD liquid medium for rejuvenation. Then, 1 mL of a resulting seed culture was transferred to 50 mL of a BMGY medium and cultured for 18 h to 24 h under shaking at 28° C. and 200 rpm to 250 rpm. 40 mL of a resulting seed culture was inoculated at an inoculum size of 4% into 1,000 mL of a BMGY medium and cultured for 24 h to 30 h under shaking at 28° C. and 250 rpm to 300 rpm. A resulting culture was then centrifuged at 5,000 rpm for 5 min. A resulting supernatant was discarded. A resulting cell pellet was resuspended in 500 mL of a BMMY induction medium and further cultured for 3 d to 4 d under shaking at 28° C. and 250 rpm to 300 rpm, during which 100% methanol was supplemented to a final concentration of 0.5% every 24 h. At this point, a cell density of the Pichia pastoris engineered strain typically reached OD₆₀₀ of 18 to 20, and the exogenous protein expressed by the gene was predominantly secreted into the liquid medium. A resulting fermentation broth was centrifuged at 10,000 rpm and 4° C. for 20 min. A resulting cell pellet was discarded. A resulting supernatant including a substantial amount of the HSA product was collected and analyzed by SDS-PAGE. This small-scale induction experiment was used to analyze and identify the expression level of the recombinant HSA.

If the high-level expression engineered strain was a Pichia pastoris CBS7435 strain, a small-scale induction expression experiment was conducted according to the following experimental protocol: glycerol stock→YPD→YPD→BSM→BSM. Inoculum sizes were similar to those for the GS115 strain.

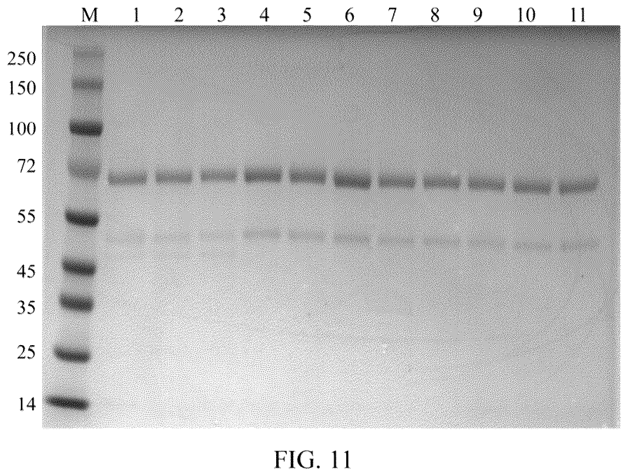

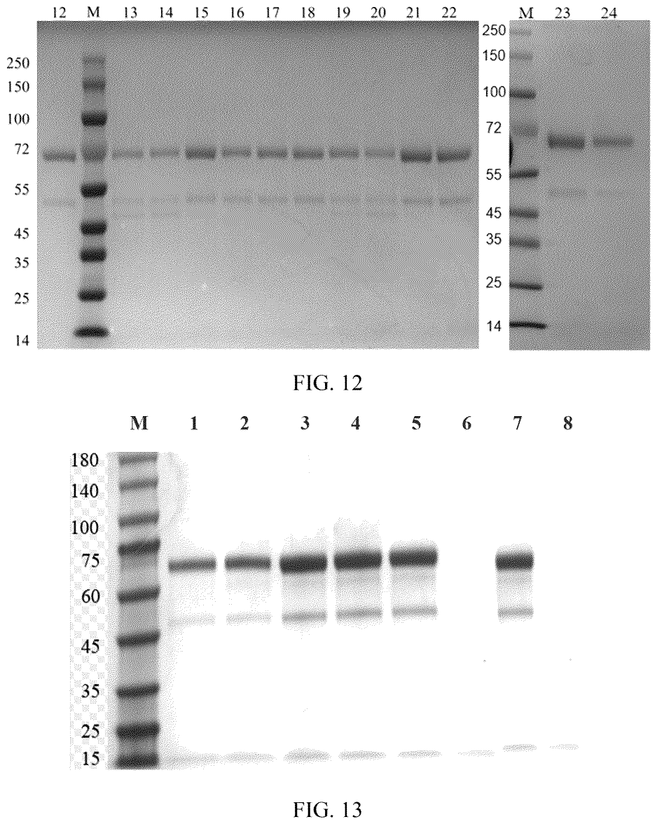

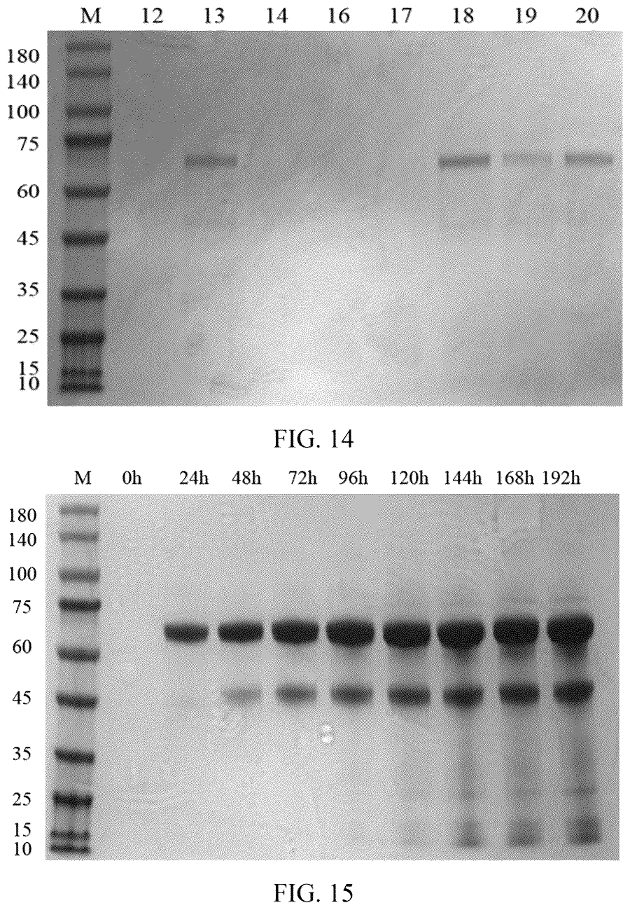

Results of the small-scale induction experiment for the Pichia pastoris GS115 or CBS7435 strain in this example are partially shown in FIG. 11, FIG. 12, FIG. 13, and FIG. 14.

An engineered strain with a distinct expression band during the small-scale induction experiment was further subjected to a high-cell-density fermentation experiment with 5 L, 30 L, 50 L, and 500 L fermenters in a fed-batch-fermentation manner, which was intended to investigate the Pichia pastoris BSM fermentation process and further validate the pilot-scale fermentation process for the GS115 or CBS7435 engineered strain undergoing electroporation. A specific implementation process included the following steps:

### S210. Medium Preparation.

The preparation of a fermentation substrate medium in this example was shown in Table 3 below.

TABLE 3
Preparation of the fermentation substrate medium (6 L)

| Reagent | Formula (g/L) | Preparation volume: 6 L |
|---|---|---|
| H₃PO₄ | 26.7 ml | 160 ml |
| CaSO₄•2H₂O | 0.47 | 2.8 |
| K₂SO₄ | 9.1 | 55 |
| MgSO₄•7H₂O | 7.5 | 45 |
| KOH | 4.13 | 25 |
| 98% glycerol | 40 | 240 |
| Defoaming agent | 0.5 ml | 3 ml |

During the preparation of the fermentation substrate medium, phosphoric acid and glycerol should be stored separately in graduated cylinders or beakers. Other inorganic salts should be dissolved sequentially with approximately 2 L of purified water. That is, after the previous reagent is fully dissolved, the next reagent is then added. KOH should also be dissolved separately and should not be mixed with the inorganic salts.

### S220. Electrode Calibration, Including pH Electrode Calibration and Dissolved Oxygen (DO) Electrode Calibration.

pH electrode calibration: Standard buffers of pH 6.86 and pH 4.00 for a pH electrode, a washing bottle, and a paper towel were first prepared. The pH electrode was connected to an electrode wire of a fermenter, rinsed with the washing bottle, blot-dried with the paper towel, and then immersed in the standard buffer of pH 6.86. Once the pH value of the fermenter was stabilized, a calibration button was pressed for zero-point calibration. The pH electrode was taken out, rinsed with the washing bottle, blot-dried with the paper towel, and then immersed in the standard buffer of pH 4.00. Similarly, once a pH value displayed was stabilized, a calibration button was pressed for slope calibration. This operation was repeated three times. When a pH value displayed was consistent with a pH value of the standard buffer, it indicated that the calibration for the pH electrode was completed.

DO electrode calibration: A DO electrode was connected to an electrode wire of the fermenter, cleaned, immersed in a saturated anhydrous sodium sulfite solution, and allowed to stand for 15 min until a displayed DO value no longer changed. Then, the calibration button was pressed for dissolved-oxygen-zero-point calibration. For the slope calibration of the DO electrode, before inoculation for fermentation, stirring was conducted at 100 rpm, and aeration was conducted at 0.5 vvm. Under a pressure of 0.05 MPa, calibration was conducted with a DO saturation point as 100% to determine a slope, thereby completing the calibration for the DO electrode.

### S230. Feeding.

Feeding process: Phosphoric acid and glycerol were fed successively into the fermenter, then an inorganic salt solution was added, and then KOH was added. Purified water was added to 5 L (a reference weight of the fermenter was approximately 5 kg, and a final volume of the reaction system after sterilization should be about 6 L). After the feeding was completed, the pH value should be 2 or lower. 1 L of PTM₁ was prepared, and sealed and stored in the dark.

3 L of 100% analytical-grade methanol was fed.

800 mL of a 50% glycerol feed was prepared as follows:

400 g of 98% glycerol was weighed, and purified water was added to 800 mL. After dissolution, a resulting solution was then transferred to a 1 L feed bottle, autoclaved at 121° C. for 30 min, and cooled for later use.

### S240. Inoculation: The Host Strain Highly Expressing the HSA was Inoculated.

Fermentation process of a Pichia pastoris engineered strain

### 1. Preparation of a Seed Culture:

A glycerol stock numbered CBS7435-C1-C50 or GS115-G1-G30 was taken. 100 μL to 20 mL of the glycerol stock was inoculated into 100 mL of a YPD medium as a primary seed culture, and cultured for 24 h under shaking at 28° C. and 200 rpm. Then, 5 mL of the glycerol stock was inoculated into 480 mL of a YPD medium in a 2 L Erlenmeyer flask as a primary seed culture, and further cultured under shaking at 28° C. and 200 rpm for 24 h. 5 mL of the primary seed culture was inoculated into 480 mL of a BSM medium in a 2 L Erlenmeyer flask (two flasks in total) as a secondary seed culture, and further cultured for 24 h under shaking at 28° C. and 200 rpm. OD₆₀₀ of the secondary seed culture should be 20 to 40 prior to inoculation.

Inoculation of a Seed Culture into a Fermenter:

Before inoculation, a temperature was set to 30° C. and a pH was set to 5.0 in the fermenter. The pH was automatically adjusted with ammonia water. Then, the seed culture was inoculated into the fermenter by the flame loop inoculation method. Flame loop inoculation: An inoculation port of the fermenter was surrounded with an alcohol cotton ball and ignited. A gas inlet valve was turned down and an exhaust valve was turned up to adjust the pressure in the fermenter to approximately 0.02 MPa. The inoculation port was slightly opened with a special tool, such that a small amount of a gas was discharged until there was no sound of air outflow. Then, the inoculation port was fully unscrewed. Packing ropes for a seed culture bottle and a PTM₁ container were removed, sealing films were then removed above a flame loop, and the seed culture and PTM₁ were then poured into the seed culture bottle and the PTM₁ container, respectively. The seed culture and the PTM₁ should not be mixed before inoculation.

### Fermentation Stage for Recombinant HSA:

### (1) Basic Culture Stage of an Engineered Strain

When a fermentation process of the engineered strain was started, the SCADA process control software was initiated for data recording, fermentation time recording, and batch number recording, and chart recording was started. After the seed culture was inoculated into the fermenter, relevant fermentation data (such as time, temperature, stirring, pH, dissolve oxygen (DO), air flow rate, and pressure) was recorded regularly at an interval of 4 h. Control parameters were set as follows: The pH was controlled at 5.5±0.1 with ammonia water. The temperature was controlled at 28±1° C. The DO value was controlled at 30 or more. The air flow rate was controlled at 2 vvm. The pressure was controlled at 0.03 MPa to 0.04 MPa. After a DO rebound (the DO rebound referred to a DO value increase of 10 or more without any adjustment to the aeration, stirring, or pressure for the fermenter), a transitional culture stage was reached. When there was a DO rebound, OD₆₀₀ of a sample collected should be approximately 50.

### (2) Transitional Culture Stage of the Engineered Strain

The transitional culture stage of the engineered strain was to further increase the biomass of the engineered strain. Fermentation data was recorded hourly. At the transitional culture stage, 50% glycerol needed to be fed. After the DO rebound, 50% glycerol was immediately fed (a PTM₁ solution including biotin was also fed at 12 mL/L, with approximately 10 mL in total). A rotational speed for the stirring was adjusted to a maximum rotational speed for the fermenter, the air flow rate was adjusted to 2 vvm, and the pressure was adjusted to 0.04 MPa to 0.08 MPa. Under these conditions, a feeding rate was controlled to maintain the DO value at 30±10. At least one DO-Spike test was conducted every hour, ensuring that a DO-Spike response time was 60 s or less. A feeding cycle was approximately 4 h to 5 h. During the feeding of glycerol, a set pH value was evenly adjusted every hour, such that a pH at the end of the glycerol feeding was an induction pH required by this project (for this fermentation, a pH at an induction stage and a pH at a culture stage were the same and were both 5.7, and thus the pH adjustment was not required). At the end of the transitional culture stage, OD₆₀₀ was about 100. At this point, a sample was collected and measured for a wet cell weight, and the sample was denoted as a 0 h sample. Subsequently, after each sampling, only a wet cell weight was measured, and OD₆₀₀ was not detected. The glycerol feeding was stopped, and a methanol induction stage was reached.

### (3) Induced Expression Stage of the Engineered Strain

After the glycerol feeding was stopped, when a DO rebound was observed, methanol needed to be fed (12 mL/L of PTM₁ was added to methanol). 4 g/L of methanol was fed at one time (for this experiment, 24 g of methanol was added at one time, and 30 mL of methanol could be fed using a disposable syringe through a feed septum). After the added methanol was depleted (indicated by a DO rebound), methanol was fed in a DO-Spike manner. A DO-Spike response time of 60 s or less was preferred, and a DO value should be controlled at 10 to 30. When the feeding of methanol was started, a temperature was adjusted to a temperature value specified by this project (for this experiment, an induction temperature was 24° C.) and a pH was controlled at 5.7 specified by this project. At the induced expression stage of the engineered strain, a DO-Spike test was conducted every 12 h, and a response time and other fermentation data were recorded. A sample was collected every 12 h, and tested for a pH and a wet cell weight. Centrifugation was conducted. 100 μL of a resulting supernatant was collected, an electrophoresis buffer was added, and a resulting mixture was boiled at 100° C. for 5 min and stored at −20° C. The remaining supernatant was stored in a 2° C. to 8° C. refrigerator for later testing or electrophoretic analysis. All samples collected were properly labeled. Throughout the induced expression stage of the Pichia pastoris engineered strain, a methanol concentration of the medium in the fermenter was maintained at 0.2% to 0.9%.

### (4) Harvesting

For the fermenter-based fermentation experiment of the Pichia pastoris engineered strain, an induced expression duration was approximately 200 h. After the induced expression was completed, a fermentation broth was harvested and subjected to solid-liquid separation and a post-treatment. A supernatant was collected for subsequent purification.

The detection of the target protein during the fermentation experiment of the Pichia pastoris engineered strain was as follows:

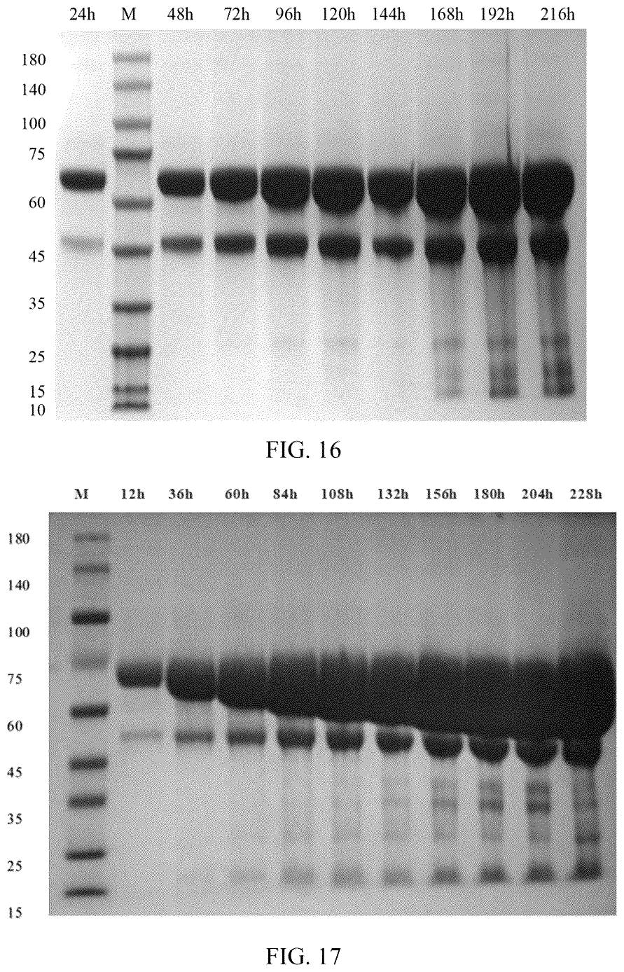

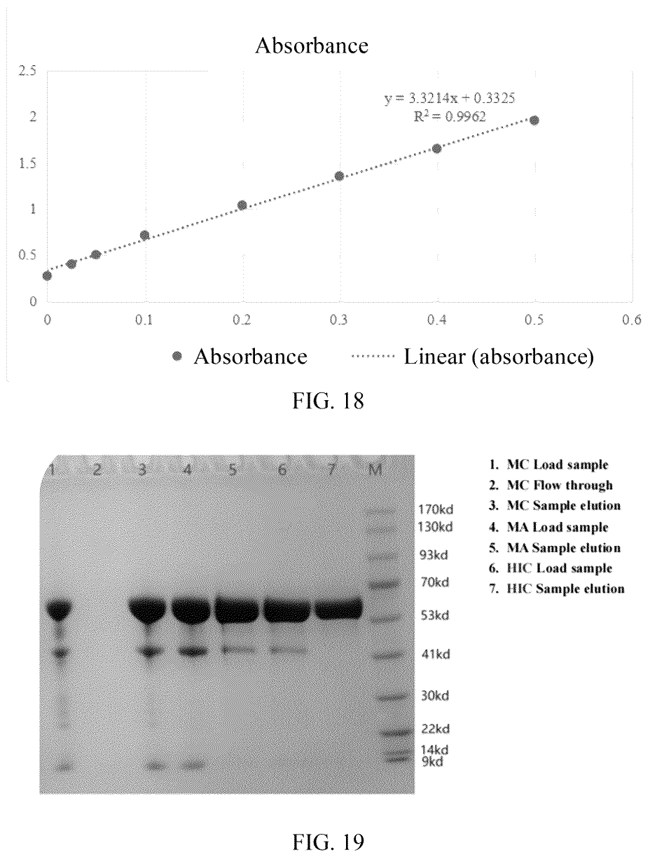

In this example, the online process control and real-time monitoring were implemented for a fermentation process of the Pichia pastoris engineered strain in the fermenter. Particularly, the target protein in a fermentation broth in the fermenter was tracked and detected on-line at different time points of the induced expression stage. In the present disclosure, an enhanced BCA protein assay kit from Beyotime Biotechnology was adopted as a detection tool. The enhanced BCA protein assay kit, developed based on the BCA method as one of the two protein concentration detection methods most widely used worldwide, achieved the high stability, high sensitivity, and high compatibility in protein concentration measurements. In this experiment, with bovine serum albumin as a standard, a standard curve and a linear equation thereof were established (as shown in FIG. 18) to enable the quantitative analysis for an expression level of HSA secreted in each batch of a fermentation broth in the fermenter. A specific process was as follows: At different time points of the induced expression stage, a fermentation broth in the fermenter was sampled and centrifuged (5,000 g×20 min). A resulting cell pellet was discarded, and a resulting supernatant was retained. After the fermentation of this batch was completed, the retained fermentation supernatant samples were collectively subjected to SDS-PAGE, and then stained with Coomassie Brilliant Blue. The protein electrophoresis results were recorded in FIG. 15, FIG. 16, and FIG. 17. In addition, the retained fermentation supernatant samples were appropriately diluted and added to a 96-well plate, and a chromogenic agent was added. Each sample was detected according to the instructions of the enhanced BCA protein assay kit. Expression levels of HSA at different time points during a fermentation process were calculated. Partial results were listed in Tables 4 and 5. Additionally, test results of expression levels of recombinant HSA from 9 other batches of fermentation were shown in Table 6.

TABLE 4
Measured concentrations of the target protein in supernatants collected at different time
points during a 50 L fermenter-based fermentation experiment of the engineered strain C7

| Induction time | 24 h | 48 h | 72 h | 96 h | 120 h | 144 h | 168 h | 192 h | 216 h | 240 h | 264 h |
|---|---|---|---|---|---|---|---|---|---|---|---|
| Measured | 0.388 | 0.392 | 0.458 | 0.511 | 0.579 | 0.696 | 0.753 | 0.842 | 0.947 | 1.048 | 1.098 |
| absorbance | | | | | | | | | | | |
| value | | | | | | | | | | | |
| Protein | 1.67 | 1.79 | 3.78 | 5.37 | 7.42 | 10.94 | 12.66 | 15.34 | 18.50 | 21.54 | 23.05 |
| concentration | | | | | | | | | | | |
| (g/L) | | | | | | | | | | | |

TABLE 5
Measured concentrations of the target protein in supernatants collected at different time
points during a 50 L fermenter-based fermentation experiment of the engineered strain C1

| Induction time | 24 h | 48 h | 72 h | 96 h | 120 h | 144 h | 168 h | 192 h | 216 h | 240 h | 264 h |
|---|---|---|---|---|---|---|---|---|---|---|---|
| Measured | 0.340 | 0.384 | 0.463 | 0.539 | 0.644 | 0.743 | 0.710 | 0.749 | 0.821 | 0.915 | 1.036 |
| absorbance | | | | | | | | | | | |
| value | | | | | | | | | | | |
| Protein | 0.23 | 1.55 | 3.93 | 6.22 | 9.38 | 12.36 | 11.37 | 12.54 | 14.71 | 17.54 | 21.18 |
| concentration | | | | | | | | | | | |
| (g/L) | | | | | | | | | | | |

TABLE 6
Results of multi-batch production of recombinant
HSA using a high-density fermentation technology

| | | | Methanol | | HSA |
|---|---|---|---|---|---|
| | | | induction | Absorbance | expression |
| Batch | Strain | pH | time (h) | (A562) | level (g/L) |
| 1 | C1 | 5.70 | 264 | 1.036 | 21.18 |
| 2 | C3 | 5.71 | 204 | 0.920 | 17.69 |
| 3 | C6 | 5.70 | 204 | 1.190 | 25.82 |
| 4 | C7 | 5.73 | 264 | 1.086 | 23.05 |
| 5 | C18 | 5.74 | 204 | 0.956 | 18.77 |
| 6 | C20 | 5.72 | 204 | 1.016 | 20.58 |
| 7 | G6 | 5.70 | 180 | 0.583 | 7.54 |
| 8 | G15 | 5.71 | 180 | 0.616 | 5.58 |
| 9 | G21 | 5.73 | 180 | 0.525 | 5.80 |

In Table 6, “C” represents the engineered strain CBS7435 and “G” represents the engineered strain GS115.

The step S300 was specifically as follows:

S310. A fermentation broth was harvested.

S320. The fermentation broth was centrifuged.

S330. A resulting fermentation supernatant was subjected to membrane filtration.

S340. A heat treatment was conducted.

S350. Membrane filtration was conducted once again.

S360. Chromatography was conducted.

The step S310 was specifically as follows: The fermentation broth was collected from the fermenter after the fermentation in the step S2 was completed.

The step S320 was specifically as follows: The fermentation broth collected in the step S310 was centrifuged at 9,000 rpm and 30° C. or lower.

The step S330 was specifically as follows: The fermentation supernatant produced after the centrifugation in the step S320 was filtered with a hollow fiber membrane to produce a filtered fermentation supernatant.

The step S340 was specifically as follows: Sodium caprylate was added at a final concentration of 5 mmol/L to the filtered fermentation supernatant obtained in the step S330, a pH was adjusted to 5.8 to 7.0, and the heat treatment was conducted at 68° C. for 30 min. A resulting system was then cooled rapidly and adjusted with acetic acid to a pH of 4.5 to produce a heat-treated fermentation supernatant.

The step S350 was specifically as follows: The heat-treated fermentation supernatant obtained in the step S340 was filtered with a hollow fiber membrane once again to produce a fermentation supernatant to be purified.

The step S360 was specifically as follows: The fermentation supernatant to be purified obtained in the step S350 was subjected to column chromatography to produce purified recombinant HSA.

The column chromatography is selected from:
a three-step column chromatography process including TH-MC mixed-mode SP cation exchange column chromatography, hydroxyapatite II (HAP II) column chromatography, and DEAE anion exchange column chromatography;
or
a three-step column chromatography process including TH-MC mixed-mode SP cation exchange column chromatography, HAP II column chromatography, and TA-phenyl-HIC hydrophobic column chromatography.

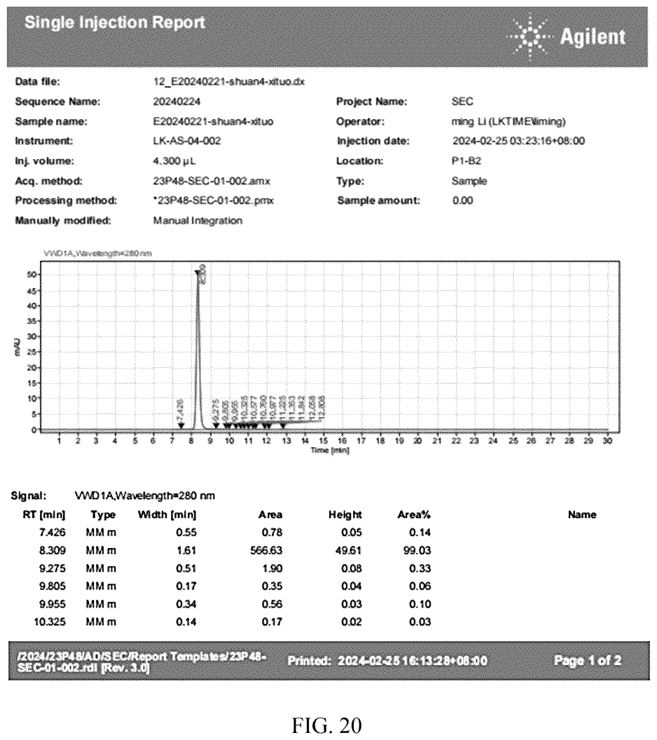

The purified recombinant HSA sample was subjected to gel electrophoresis, and a single band appeared, as shown in FIG. 19. The purity of the recombinant HSA sample was determined by high-performance liquid chromatography (HPLC) analysis, and the purity reached 99.03% with a single peak, as shown in FIG. 20.

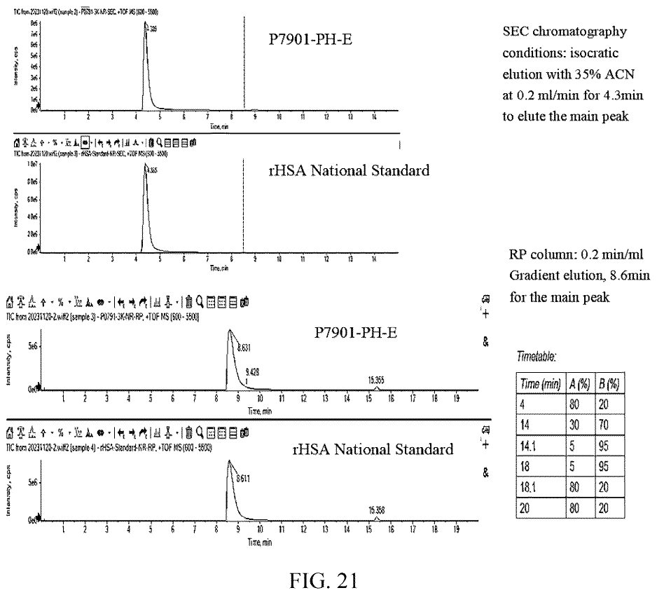

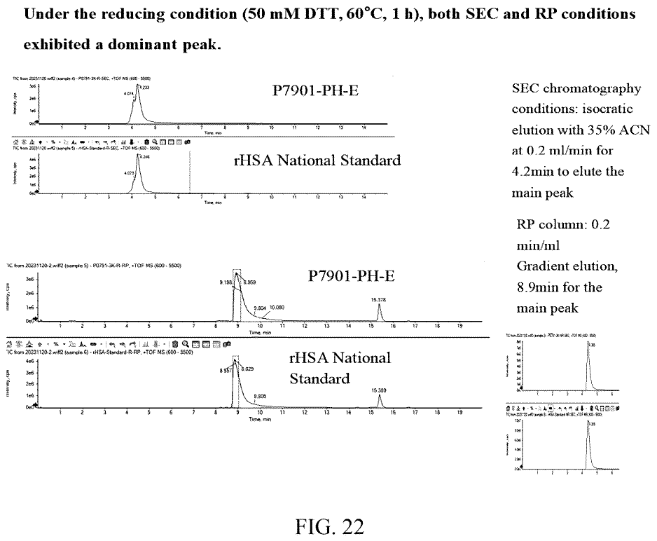

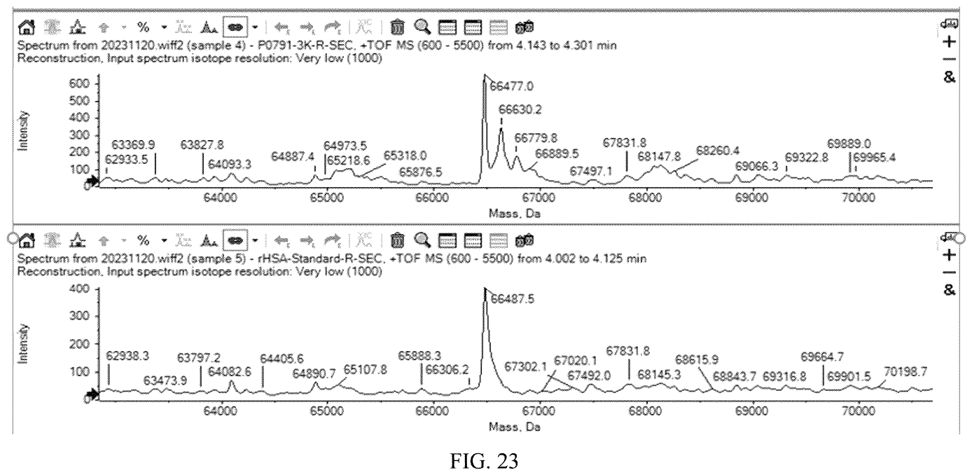

The purified recombinant HSA sample (No. P7901-PH-E) and the rHSA national standard (purchased from the National Institutes for Food and Drug Control, China) were preliminarily subjected to molecular weight characterization using mass spectrometry. Under both non-reducing and reducing conditions, the comparative analysis was conducted using mass spectrometry with size-exclusion chromatography (SEC) and reverse-phase (RP) elution. TIC results of the purified recombinant HSA sample and the rHSA national standard under the above conditions all showed single peaks, and were highly consistent in terms of a peak time and pattern (as shown in FIG. 21 and FIG. 22). Further, a molecular weight was determined by electrospray ionization mass spectrometry. Results revealed that the measured molecular weights of the recombinant HSA sample (No. P7901-PH-E) and the rHSA national standard were 66,477.0 Da and 66,487.5 Da, respectively, which were both highly consistent with the theoretical molecular weight of 66,472 Da for HSA, as shown in FIG. 23.

This example provides a recombinant expression vector for producing highly-expressed HSA, a host strain, and a use of the host strain. The constructed recombinant expression vector and host strain have the following beneficial effects:

1) Through the molecular-level optimization design for the full sequence of the target gene, a target gene fragment most suitable for the expression in Pichia pastoris is acquired. 2) At the cellular level, the internationally latest Pichia pastoris expression system host strain CBS7435 is introduced to express the gene for HSA. Pichia pastoris transformants carrying the exogenous gene with a high copy number are selected with the G418 resistance gene to construct a Pichia pastoris engineered strain for expressing recombinant HSA at a high level. 3) At the engineering level, optimized solutions for a fermentation culture process and reactor process control are explored to further improve the expression level of the target protein. 4) In terms of the outstanding shortcomings of the existing purification process for recombinant HSA, the latest purification media, technologies, and approaches inside and outside China are introduced. The purification process of the present disclosure involves only pretreatment steps such as solid-liquid separation, heat treatment, and ultrafiltration concentration for a fermentation broth, and can achieve the entire purification merely through three-step column chromatography. A purity of the recombinant HSA reaches 99% or higher. A recovery rate of the entire purification process of the present disclosure is 60% or higher.

The above examples merely represent several implementations of the present disclosure, and the descriptions thereof are specific and detailed. However, these examples should not be construed as limiting the patent scope of the present disclosure. It should be noted that those of ordinary skill in the art can further make several variations and modifications without departing from the concept of the present disclosure, and such variations and modifications all fall within the claimed scope of the present disclosure. Therefore, the protection scope of the present disclosure shall be subject to the protection scope defined by the claims.

What is claimed is:

1. A recombinant expression vector for producing highly-expressed human serum albumin (HSA), wherein the nucleotide sequence of the recombinant expression vector is shown in SEQ ID NO: 3.

2. The recombinant expression vector for producing the highly-expressed HSA according to claim 1, comprising:
(1) a 5′ regulatory region, wherein the 5′ regulatory region is derived from Pichia pastoris and comprises a promoter element, the 5′ regulatory region is selected from a 5′ regulatory region of an alcohol oxidase 1 (AOX1) gene, a dihydroxyacetone synthase (DAS1) gene, or a histidinol dehydrogenase (HIS4) gene, wherein the AOX1 gene, the DAS1 gene, or the HIS4 gene is derived from the Pichia pastoris, and a 3′ terminus of the 5′ regulatory region is linked to a sequence described in (2) below;
(2) an optimized gene encoding HSA, wherein the nucleotide sequence of the optimized gene is shown in SEQ ID NO: 1, and the amino acid sequence of the HSA is shown in SEQ ID NO: 2; and
(3) a 3′ termination sequence derived from the Pichia pastoris, wherein the 3′ termination sequence is selected from a 3′ termination sequence of the AOX1 gene, an alcohol oxidase 2 (AOX2) gene, or the HIS4 gene, wherein the AOX1 gene, the AOX2 gene, or the HIS4 gene is derived from the Pichia pastoris.

3. The recombinant expression vector for producing the highly-expressed HSA according to claim 2, further comprising:
at least one marker gene for screening in Escherichia coli (E. coli);
a DNA fragment of a replication origin capable of replicating in an E. coli host strain; and
at least two marker genes for screening in a yeast.

4. The recombinant expression vector for producing the highly-expressed HSA according to claim 2, wherein the recombinant expression vector is a linearized vector produced by cleaving one of pPIC9, pPIC3, pPICZαABC, pPIC3.5K, pHIL-S1, pHIL-D2, pA0804, pA0815, pGAPZαABC, pPIC6αABC, and pPIC9K with a restriction endonuclease SacI or a restriction endonuclease BglII.

5. The recombinant expression vector for producing the highly-expressed HSA according to claim 4, wherein the recombinant expression vector is a linearized vector produced by cleaving the pPIC9K with the restriction endonuclease SacI or the restriction endonuclease BglII, and the linearized vector produced by cleaving the pPIC9K with the restriction endonuclease SacI or the restriction endonuclease BglII comprises at least one copy of the optimized gene encoding the HSA.

6. The recombinant expression vector for producing the highly-expressed HSA according to claim 2, wherein in the optimized gene encoding the HSA, optimal codons preferred by the AOX1 gene derived from the Pichia pastoris are adopted, and a proportion of the optimal codons in total codons of the optimized gene is controlled at 90%.

7. The recombinant expression vector for producing the highly-expressed HSA according to claim 2, wherein three restriction endonuclease sites SalI, HindIII, and XbaI are inserted sequentially in a 5′ to 3′ direction within the optimized gene encoding the HSA, such that the optimized gene is divided into four relatively-balanced large fragments.

8. The recombinant expression vector for producing the highly-expressed HSA according to claim 2, wherein in the optimized gene encoding the HSA, consecutive G-C base pairs are reduced and A-T base pairs preferred by the Pichia pastoris are increased, such that a GC content in the optimized gene is adjusted to 45% to 50%.

9. The recombinant expression vector for producing the highly-expressed HSA according to claim 2, wherein the optimized gene encoding the HSA comprises an expression reading cassette constructed as follows:
inserting a 5′ restriction endonuclease site BamHI into the 5′ regulatory region of the AOX1 gene, ligating the 10-deoxynucleotide oligonucleotide CCAAACGATG as shown in SEQ ID NO: 9 comprising a Kozak sequence for eukaryotic genes, ligating a yeast α-mating factor leader peptide sequence comprising 85 amino acids derived from Saccharomyces cerevisiae, and inserting a mature HSA gene between multiple cloning sites EcoRI and Not of a pPIC9K recombinant expression vector, wherein the 5′ regulatory region is a promoter region, and the Kozak sequence for the eukaryotic genes is AXXATG; and
inserting a coding sequence AAAAGA for a dibasic amino acid of lysine and arginine after an enzyme cleavage site EcoRI at a 5′ terminus of the mature HSA gene to obtain the sequence as shown in SEQ ID NO: 4, and inserting a double stop codon TAATAG before an enzyme cleavage site NotI at a 3′ terminus of the mature HSA gene to obtain the sequence as shown in SEQ ID NO: 5.

10. A host strain for high-level expression of HSA, wherein the host strain is Pichia pastoris HSA-C16, wherein the Pichia pastoris HSA-C16 is deposited at China General Microbiological Culture Collection Center (CGMCC) with an accession number of CGMCC No. 30175, and a deposit date is Mar. 28, 2024.

11. The host strain for the high-level expression of the HSA according to claim 10, wherein the host strain is a recombinant HSA-expressing engineered strain constructed by introducing a recombinant expression vector for producing highly-expressed HSA into a Pichia pastoris strain CBS7435, wherein the nucleotide sequence of the recombinant expression vector is shown in SEQ ID NO: 3.

12. A method for preparing HSA, comprising using the host strain for the high-level expression of the HSA according to claim 10.

13. The host strain for the high-level expression of the HSA according to claim 11, wherein the recombinant expression vector comprises:
(1) a 5′ regulatory region, wherein the 5′ regulatory region is derived from Pichia pastoris and comprises a promoter element, the 5′ regulatory region is selected from a 5′ regulatory region of an AOX1 gene, a DAS1 gene, or an HIS4 gene, wherein the AOX1 gene, the DAS1 gene, or the HIS4 gene is derived from the Pichia pastoris, and a 3′ terminus of the 5′ regulatory region is linked to a sequence described in (2) below;
(2) an optimized gene encoding HSA, wherein the nucleotide sequence of the optimized gene is shown in SEQ ID NO: 1, and the amino acid sequence of the HSA is shown in SEQ ID NO: 2; and
(3) a 3′ termination sequence derived from the Pichia pastoris, wherein the 3′ termination sequence is selected from a 3′ termination sequence of the AOX1 gene, an AOX2 gene, or the HIS4 gene, wherein the AOX1 gene, the AOX2 gene, or the HIS4 gene is derived from the Pichia pastoris.

14. The host strain for the high-level expression of the HSA according to claim 13, wherein the recombinant expression vector further comprises:
at least one marker gene for screening in E. coli;
a DNA fragment of a replication origin capable of replicating in an E. coli host strain; and
at least two marker genes for screening in a yeast.

15. The host strain for the high-level expression of the HSA according to claim 13, wherein the recombinant expression vector is a linearized vector produced by cleaving one of pPIC9, pPIC3, pPICZαABC, pPIC3.5K, pHIL-S1, pHIL-D2, pA0804, pA0815, pGAPZαABC, pPIC6αABC, and pPIC9K with a restriction endonuclease SacI or a restriction endonuclease BglII.

16. The host strain for the high-level expression of the HSA according to claim 15, wherein the recombinant expression vector is a linearized vector produced by cleaving HSA-pPIC9K with the restriction endonuclease SacI or the restriction endonuclease, and the linearized vector produced by cleaving the HSA-pPIC9K with the restriction endonuclease SacI or the restriction endonuclease Bgl II comprises at least one copy of the optimized gene encoding the HSA.

17. The host strain for the high-level expression of the HSA according to claim 13, wherein in the optimized gene encoding the HSA of the recombinant expression vector, optimal codons preferred by the AOX1 gene derived from the Pichia pastoris are adopted, and a proportion of the optimal codons in total codons of the optimized gene is controlled at 90%.

18. The host strain for the high-level expression of the HSA according to claim 13, wherein in the recombinant expression vector, three restriction endonuclease sites SalI, HindIII, and XbaI are inserted sequentially in a 5′ to 3′ direction within the optimized gene encoding the HSA, such that the optimized gene is divided into four relatively-balanced large fragments.

19. The host strain for the high-level expression of the HSA according to claim 13, wherein in the optimized gene encoding the HSA of the recombinant expression vector, consecutive G-C base pairs are reduced and A-T base pairs preferred by the Pichia pastoris are increased, such that a GC content in the optimized gene is adjusted to 45% to 50%.

20. The host strain for the high-level expression of the HSA according to claim 13, wherein in the recombinant expression vector, the optimized gene encoding the HSA comprises an expression reading cassette constructed as follows:
inserting a 5′ restriction endonuclease site BamHI into the 5′ regulatory region of the AOX1 gene, ligating the 10-deoxynucleotide oligonucleotide CCAAACGATG as shown in SEQ ID NO: 9 involving a Kozak sequence for eukaryotic genes, ligating a yeast α-mating factor leader peptide sequence comprising 85 amino acids derived from Saccharomyces cerevisiae, and inserting a mature HSA gene between multiple cloning sites EcoRI and NotI of a pPIC9K recombinant expression vector, wherein the 5′ regulatory region is a promoter region, and the Kozak sequence for the eukaryotic genes is AXXATG; and
inserting a coding sequence AAAAGA for a dibasic amino acid of lysine and arginine after an enzyme cleavage site EcoRI at a 5′ terminus of the mature HSA gene to obtain the sequence as shown in SEQ ID NO: 4, and inserting a double stop codon TAATAG before an enzyme cleavage site NotI at a 3′ terminus of the mature HSA gene to obtain the sequence as shown in SEQ ID NO: 5.


SEQUENCE LISTING (9 sequences)

SEQ ID NO: 1 - DNA, 1755 bp, synthetic construct
  source 1..1755: mol_type = other DNA
gatgcgcacaaatcagaggtggcgcataggttcaaggaccttggagaagaaaacttcaaggctctagtattgatagcatttgcacaatacttacaacagtgtccatttgaagaccacgtaaaactcgttaatgaggtcactgagttcgccaagacctgcgtggccgatgaatcggcggagaattgtgataaaagtcttcacactcttttcggcgacaagctatgcactgtagcgacattgcgagaaacctacggtgagatggcggattgctgtgccaagcaggagcctgagcgcaatgagtgttttttgcagcacaaagatgacaaccccaatcttcccaggttggtgcggcccgaagttgacgttatgtgcacggcgtttcatgacaacgaagagacttttctgaagaagtatctatatgaaatagcacggagacacccatatttttacgcccctgagctgctcttttttgctaagaggtataaggcggcttttacagaatgttgtcaggcagcggataaggccgcgtgcctcttacccaagttggacgaacttcgagatgaagggaaggcgtctagtgccaagcaacgtctcaagtgcgcatcactccagaagttcggagagagagccttcaaagcttgggcggtggcccgactatcccagcgctttccgaaagctgagttcgcagaggtatcgaagctcgtaaccgaccttacgaaagtacatactgaatgttgccatggtgacttgctggagtgcgcggatgacagagctgatctagcgaaatacatatgtgaaaaccaagatagcattagtagcaagctgaaagaatgctgcgaaaaacccttgttggagaagtctcattgcattgctgaagtagaaaatgacgagatgccggcagatcttccctccttagccgcggatttcgtcgagagcaaggacgtttgtaaaaattacgcagaagccaaggatgtgttcctagggatgtttctgtacgagtatgcccgtcgtcacccggattattcagtcgtcctgctgctccggttagccaaaacatacgagactaccttagaaaaatgctgtgctgccgctgacccacacgaatgctacgctaaggtctttgacgaatttaaaccgctggtggaggagcctcaaaacctcatcaaacagaattgtgaactcttcgaacagctaggagagtacaagttccaaaacgctctactggtgagatatacaaaaaaggtgccgcaagtctcgacaccaacattagtcgaagtttcgcggaaccttggcaaagttggttccaaatgttgcaagcatcctgaagcaaaaaggatgccatgcgcagaagactatttatcagttgtacttaaccaactttgtgtcttgcacgaaaaaactccggtatccgaccgtgtcacgaaatgttgtacggagagcctggtaaaccgccgaccttgcttcagtgctctcgaggtggatgagacctatgtcccaaaagaatttaatgcagaaacgtttaccttccatgcagatatctgtaccctatctgaaaaagagcgccaaattaagaagcaaacggcgcttgttgagttagttaaacataaacctaaagcaacgaaagagcagttaaaggctgtgatggacgacttcgcggcatttgtcgagaaatgctgcaaggctgatgacaaagagacatgttttgccgaggaaggcaagaaactcgttgccgcttctcaggcagccctagggtta

SEQ ID NO: 2 - protein, 585 aa, synthetic construct [sequence omitted]

SEQ ID NO: 3 - DNA, 11037 bp, synthetic construct
  source 1..11037: mol_type = other DNA
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcttacgtagaattcaaaagagatgcgcacaaatcagaggtggcgcataggttcaaggaccttggagaagaaaacttcaaggctctagtattgatagcatttgcacaatacttacaacagtgtccatttgaagaccacgtaaaactcgttaatgaggtcactgagttcgccaagacctgcgtggccgatgaatcggcggagaattgtgataaaagtcttcacactcttttcggcgacaagctatgcactgtagcgacattgcgagaaacctacggtgagatggcggattgctgtgccaagcaggagcctgagcgcaatgagtgttttttgcagcacaaagatgacaaccccaatcttcccaggttggtgcggcccgaagttgacgttatgtgcacggcgtttcatgacaacgaagagacttttctgaagaagtatctatatgaaatagcacggagacacccatatttttacgcccctgagctgctcttttttgctaagaggtataaggcggcttttacagaatgttgtcaggcagcggataaggccgcgtgcctcttacccaagttggacgaacttcgagatgaagggaaggcgtctagtgccaagcaacgtctcaagtgcgcatcactccagaagttcggagagagagccttcaaagcttgggcggtggcccgactatcccagcgctttccgaaagctgagttcgcagaggtatcgaagctcgtaaccgaccttacgaaagtacatactgaatgttgccatggtgacttgctggagtgcgcggatgacagagctgatctagcgaaatacatatgtgaaaaccaagatagcattagtagcaagctgaaagaatgctgcgaaaaacccttgttggagaagtctcattgcattgctgaagtagaaaatgacgagatgccggcagaccttccctccttagccgcggatttcgtcgagagcaaggacgtttgtaaaaattacgcagaagccaaggatgtgttcctagggatgtttctgtacgagtatgcccgtcgtcacccggattattcagtcgtcctgctgctccggttagccaaaacatacgagactaccttagaaaaatgctgtgctgccgctgacccacacgaatgctacgctaaggtctttgacgaatttaaaccgctggtggaggagcctcaaaacctcatcaaacagaattgtgaactcttcgaacagctaggagagtacaagttccaaaacgctctactggtgagatatacaaaaaaggtgccgcaagtctcgacaccaacattagtcgaagtttcgcggaaccttggcaaagttggttccaaatgttgcaagcatcctgaagcaaaaaggatgccatgcgcagaagactatttatcagttgtacttaaccaactttgtgtcttgcacgaaaaaactccggtatccgaccgtgtcacgaaatgttgtacggagagcctggtaaaccgccgaccttgcttcagtgctctcgaggtggatgagacctatgtcccaaaagaatttaatgcagaaacgtttaccttccatgcagatatctgtaccctatctgaaaaagagcgccaaattaagaagcaaacggcgcttgttgagttagttaaacataaacctaaagcaacgaaagagcagttaaaggctgtgatggacgacttcgcggcatttgtcgagaaatgctgcaaggctgatgacaaagagacatgttttgccgaggaaggcaagaaactcgttgccgcttctcaggcagccctagggttataataggcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagacagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtggcttccatggctctaagaccctttgattggccaaaacaggaagtgcgttccaagtgacagaaaccaacacctgtttgttcaaccacaaatttcaagcagtctccatcacaatccaattcgatacccagcaacttttgagttgctccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcagggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcg

SEQ ID NO: 4 - DNA, 12 bp, synthetic construct
  source 1..12: mol_type = other DNA
gaattcaaaaga

SEQ ID NO: 5 - DNA, 14 bp, synthetic construct
  source 1..14: mol_type = other DNA
taataggcggccgc

SEQ ID NO: 6 - DNA, 742 bp, synthetic construct
  source 1..742: mol_type = other DNA
ttattgtttataaatactactattgccagcattgctgctaaagaagaaggggtatctctcgagaaaagagaggctgaagcttacgtagaattcaaaagagatgcgcacaaatcagaggtggcgcataggttcaaggaccttggagaagaaaacttcaaggctctagtattgatagcatttgcacaatacttacaacagtgtccatttgaagaccacgtaaaactcgttaatgaggtcactgagttcgccaagacctgcgtggccgatgaatcggcggagaattgtgataaaagtcttcacactcttttcggcgacaagctatgcactgtagcgacattgcgagaaacctacggtgagatggcggattgctgtgccaagcaggagcctgagcgcaatgagtgttttttgcagcacaaagatgacaaccccaatcttcccaggttggtgcggcccgaagttgacgttatgtgcacggcgtttcatgacaacgaagagacttttctgaagaagtatctatatgaaatagcacggagacacccatatttttacgcccctgagctgctcttttttgctaagaggtataaggcggcttttacagaatgttgtcaggcagcggataaggccgcgtgcctcttacccaagttggacgaacttcgagatgaagggaaggcgtctagtgccaagcaacgtctcaagtgcgcatcactccagaagttcggagagagagccttcaaagcttggg

SEQ ID NO: 7 - DNA, 760 bp, synthetic construct
  source 1..760: mol_type = other DNA
tcaagtgcgcatcactccagaagttcggagagagagccttcaaagcttgggcggtggcccgactatcccagcgctttccgaaagctgagttcgcagaggtatcgaagctcgtaaccgaccttacgaaagtacatactgaatgttgccatggtgacttgctggagtgcgcggatgacagagctgatctagcgaaatacatatgtgaaaaccaagatagcattagtagcaagctgaaagaatgctgcgaaaaacccttgttggagaagtctcattgcattgctgaagtagaaaatgacgagatgccggcagaccttccctccttagccgcggatttcgtcgagagcaaggacgtttgtaaaaattacgcagaagccaaggatgtgttcctagggatgtttctgtacgagtatgcccgtcgtcacccggattattcagtcgtcctgctgctccggttagccaaaacatacgagactaccttagaaaaatgctgtgctgccgctgacccacacgaatgctacgctaaggtctttgacgaatttaaaccgctggtggaggagcctcaaaacctcatcaaacagaattgtgaactcttcgaacagctaggagagtacaagttccaaaacgctctactggtgagatatacaaaaaaggtgccgcaagtctcgacaccaacattagtcgaagtttcgcggaaccttggcaaagttggttccaaatgttgcaagcatcctgaagcaaaaaggatgccatgcgcagaaga

SEQ ID NO: 8 - DNA, 766 bp, synthetic construct
  source 1..766: mol_type = other DNA
atatacaaaaaaggtgccgcaagtctcgacaccaacattagtcgaagtttcgcggaaccttggcaaagttggttccaaatgttgcaagcatcctgaagcaaaaaggatgccatgcgcagaagactatttatcagttgtacttaaccaactttgtgtcttgcacgaaaaaactccggtatccgaccgtgtcacgaaatgttgtacggagagcctggtaaaccgccgaccttgcttcagtgctctcgaggtggatgagacctatgtcccaaaagaatttaatgcagaaacgtttaccttccatgcagatatctgtaccctatctgaaaaagagcgccaaattaagaagcaaacggcgcttgttgagttagttaaacataaacctaaagcaacgaaagagcagttaaaggctgtgatggacgacttcgcggcatttgtcgagaaatgctgcaaggctgatgacaaagagacatgttttgccgaggaaggcaagaaactcgttgccgcttctcaggcagccctagggttataataggcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagc

SEQ ID NO: 9 - DNA, 10 bp, synthetic construct
  source 1..10: mol_type = other DNA
ccaaacgatg